(19) United States
(12) Patent Application Publication
(10) Pub. No.: US 2011/0038924 A1
(43) Pub. Date: Feb. 17, 2011

(54) OSTEOARTHRITIS GENE THERAPY

(76) Inventor: Randal S. Goomer, La Costa, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/740,073

(22) PCT Filed: Oct. 29, 2008
(86) PCT No.: PCT/US08/81604

§ 371 Date: Aug. 3, 2010

Related U.S. Application Data
(60) Provisional application No. 61/000,924, filed on Oct. 29, 2007.

(51) Int. Cl. A61K 9/127; A61K 48/00; A61P 19/02; A61K 38/17; C07K 14/435; C07H 21/00

(52) U.S. Cl. 424/450; 514/44 R; 514/16.8; 530/350; 536/23.5

(57) ABSTRACT

The present invention relates to methods and compositions for protecting joint cartilage from degradation by inserting transgenes that increase Kras-2b expression or an effector thereof within one or more tissues of the joint. The present invention can thus be used in the treatment and/or prevention of joint diseases and disorders, including but not limited to osteoarthritis.

### CROSS-REFERENCE TO RELATED APPLICATIONS

This application claims priority from U.S. provisional application 61/000,924 filed Oct. 29, 2007. The contents of this document are incorporated herein by reference.

### TECHNICAL FIELD

The present invention relates to methods and compositions for protecting cartilage from degradation in the joints of a mammal; more specifically, the invention relates to techniques and polynucleotide constructs for treating or preventing joint diseases and disorders using in vivo gene therapy. The joint diseases and disorders comprise arthritic pathologies and autoimmune diseases, including but not limited to Osteoarthritis (OA), Rheumatoid arthritis (RA), Crystal disease, Coeliac disease, diabetes mellitus type 1 (IDDM), systemic lupus erythematosus (SLE), Sjögren's syndrome, multiple sclerosis (MS), Hashimoto's thyroiditis, Graves' disease and idiopathic thrombocytopenic purpura.

### BACKGROUND

Osteoarthritis (OA) is the most common musculoskeletal disease in humans. According to the Arthritis Foundation, OA affects 20.7 million Americans over the age of 45 years. OA is responsible for >7 million physician visits per year. Women are more commonly affected than men. Knee OA can be as disabling as any cardiovascular disease except stroke. In addition to the aged people, there are currently nearly 300,000 children in America with some form of arthritis. There are 8.4 million young adults between the ages of 18-44 who have arthritis. Musculoskeletal conditions such as OA cost the U.S. economy nearly $86.2 billion per year.

Clinically, OA is characterized by articular cartilage degradation followed by joint space narrowing. Multiple causative factors have been implicated, including: joint trauma, infection, bone diseases (osteochondritis dissecans or Paget's disease), rheumatoid arthritis, and crystal disease, bleeding dyscrasias which lead to repeated hemarthroses, Wilson's disease and a number of other systemic conditions. OA is also thought of as a disease that can occur insidiously during aging. Regardless of the underlying cause, the clinical findings in patients with osteoarthritis are almost universal. Patients typically complain of pain, stiffness, decreased range of motion, palpable grinding within the joint (crepitus), swelling and eventual joint enlargement or deformity. Macroscopically, the articular cartilage surface develops areas of focal damage and softening early in the disease process. As OA progresses, surface cartilaginous fibrillations and vertical clefts develop, and eventually there are large areas of full thickness cartilage loss with exposed, eburnated subchondral bone. Radiographically, this process is seen as progressive joint space narrowing (secondary to loss of the radiolucent articular cartilage), subchondral bony sclerosis and cyst formation, and the development of marginal osteophytes. Eventually, the cumulative effect of all of these changes leads to decreased use of the joint, muscular atrophy, and debilitating pain (Felson et al., (2000) Ann. Intern. Med., 133(8):635-646).

Microscopically, the synovial and cartilaginous tissues undergo characteristic changes as osteoarthritis progresses. These articular tissues show significantly increased cellular proliferation. Either before or concomitant with the development of surface fibrillations, the macromolecular framework of the matrix is disrupted, and the water content increases. This is accompanied by a decrease in the aggregation of proteoglycans, the concentration of aggrecans, and the length of the glycosaminoglycan chains. These changes lead to an increase in the overall permeability of the matrix which decreases the cartilage stiffness and makes it more susceptible to further biochemical and biomechanical damage.

At the molecular level, cartilage matrix degradation is orchestrated by immune and inflammatory signals initiated in the synovial cavity. Multiple molecular players, including inflammatory cytokines such as IL-1 and TNF, and, matrix metalloproteinases, such as MMP-2, 9 and 13 and aggracenases: ADAMTS4 and 5 have been implicated in this degradative process. Cascades of inflammatory cytokines and catabolic enzymes were shown to be released from the cells in the synovium to orchestrate cartilage degradation. Regardless of the initiating etiological factors, the events producing the pathological changes involve a cascade of biological processes (Malemuda et al., (2003) Cells Tissues Organs, 174:34-48).

Currently, there are no disease modifying drugs (DMDs) approved by the FDA for OA (Wieland et al., (2005) Nature Reviews Drug Discovery, 4:331-344). Pain remediation strategies include NSAIDs, COX-2 inhibitors and injections of Hyaluronan based viscosupplementation products such as SYNVISC® or SUPARTZ®. For most patients suffering from years of pain from osteoarthritis, the end result is knee replacement surgery. The lack of effective non-surgical options has led significant numbers of patients to self-medicating themselves by oral consumption of nutraceuticals such as glycosaminoglycans and chondroitin sulfate etc. These treatments are unproven and may cause unintended harm. Therefore, the need for an effective therapy for osteoarthritis is imperative. Gene therapy offers one of the very few viable therapeutic options by delivering proteins at the site of the pathology for an extended period of time.

In the laboratory, OA has been successfully modeled in rabbits by transecting the anterior cruciate ligament (ACLT). Numerous independent studies have shown that the experimentally induced OA in the rabbit demonstrates the same pathological progression as in humans. The ACLT model is incrementally degradative showing continuing and increasing lesions of cartilage matrix, diminution of cartilage GAGs and decreases in cartilage height starting from 4 to 12 weeks. This cartilage degradation progressively worsens, resulting in complete focal depletion of the cartilage layer by 12 weeks post-ACLT. The effect on subchondral bone density and osteophyte formation also parallels those observed in human OA (Takahashi, et al. (1999) Osteoarthritis and Cartilage, 7:182-190; Setton, et al., (1999) Osteoarthritis and Cartilage, 7:2-14; Goomer, et al., (2005) Clin. Orthop. Rel. Res., 434:239-245).

Ras proteins are members of the GTPase superfamily that bind GDP/GTP and possess intrinsic GTPase activity. The GDP-bound inactive state is converted to the GTP-bound active form upon stimulation by upstream activation signal such as binding of a growth factor to its receptor. Kras is a member of a family of ras GTPases which also includes N- and H-ras. The mutated form of ras genes causes oncogenic transformation by preferentially binding to the GTP moiety and forming a “constitutively activated complex.” However, the role of wild-type Kras in the process of differentiation and cellular proliferation is much more complicated. Recent data clearly demonstrate that the wild-type Kras is a potent tumor repressor and acts via mechanisms that are independent of that of the activated mutated form of the protein. For example, carcinogen induced lung cancer causes Kras activating mutations, and tumors formed in heterozygous Kras null mice (that express oncogenic Kras only) were more abundant, and larger, and were predominantly undifferentiated malignant adenocarcinomas, whereas tumors in wild-type mice (that express wild-type and oncogenic Kras) were smaller adenomas. Re-expression of wild-type Kras in tumor cell lines (that express oncogenic Kras) was also shown to inhibit their growth in vitro and their capacity to develop tumors in nude mice. The protective effect of the wild-type Kras allele suggests that the Kras proto-oncogene has tumor suppressor activity and is consistent with the conclusions of earlier studies that the dominant nature of ras oncogenes results from either overexpression of the mutant ras allele or loss of the wild-type allele.

Thus, K-ras appears to exert a dual function in that it promotes cancer development as a gain-of-function oncogene when mutated and inhibits cancer by loss of tumor suppressor activity when wild type (proto-oncogene). (James et al., (2003) Mol. Cancer. Res. 1:820-825).

Thus, ras-mediated signal transudation pathways critically regulate cellular proliferation and differentiation. Among the three mammalian Ras genes identified to date, K-Ras4B (or 2b), the predominant form of K-Ras, is unique in its C-terminal sequence and an upstream polylysine region, which are crucial for its association with plasma membrane.

In this disclosure, we report high efficiency non-viral delivery of genes carried on mammalian expression vectors in vivo in knee tissues such as the synovium, ligaments, meniscus and cartilage, where it downregulated specific transcripts that are important for cell proliferation. Furthermore, we report the serendipitous identification of Kras-2b gene product as a strong therapeutic against OA. Kras has never been previously identified as playing a possible role in OA. Kras is a member of a family of ras oncogenes which also includes N- and H-ras. N- and H-ras may also act in like manner to affect the OA pathology.

### BRIEF DESCRIPTION OF THE FIGURES

FIG. 1 shows that rabbit synovial tissues were efficiently transfected using the non-viral gene delivery cocktail, as detailed in the materials and methods section. Analysis of the relative expression of (β-galactosidase mRNAs in rabbit synovial tissues transfected with the β-gal gene carried on a mammalian expression plasmid was performed using real time PCR. The expression of the β-galactosidase gene was increased 8-39 times over baseline expression in the synovium of animals transfected with β-galactosidase.

FIG. 2 shows that Kras-2b mRNA was specifically expressed in rabbit synovial tissues transfected with the mammalian expression plasmids carrying the Kras-2b gene driven by a strong mammalian promoter. Kras-2b transcripts were increased 100-250 times over baseline expression in the tissues transfected with the Kras-2b plasmids.

FIG. 3 shows that expression of the S6 ribosomal gene (a small subunit ribosomal gene that is known to specifically index cellular proliferation) was selectively and dose-dependently down-regulated in wild-type Kras-2b transfected rabbit synovial tissue. Since S6 synthesis is directly controlled by members of the ras signal transduction cascade, FIG. 3 demonstrates that the forced over-expression of wild-type Kras-2b specifically and robustly restrains cellular proliferation.

FIG. 4 shows a histological section of β-galactosidase transfected intracapsular synovial tissues stained with eosin and X-gal (blue), demonstrating high efficiency of gene delivery gene delivery into the intracapsular tissue.

FIG. 5A shows gene delivery of β-galactosidase into the intracapsular tissues, and specifically, the meniscus. FIG. 5A is a gross image and not magnified. The menisci were efficiently transfected with the functional copy of the β-galactosidase expression vector. The blue staining is indicative of the presence of functionally active β-galactosidase enzyme. FIG. 5B shows gene delivery into the synovial fat pad surrounding the meniscus. FIG. 5B is at 2× magnification. Specific anatomic substructures, such as the fat pad surrounding the meniscus were more efficiently transfected as a result of direct injection into the knee cavity. FIG. 5C shows gene delivery into the intracapsular tissues and specifically the ACL. FIG. 5C is not magnified.

FIG. 6 shows a typical contra-lateral non-surgical control femoral condyle with typical high surface reflectance and no apparent regions of fibrillation.

FIG. 7 shows a typical femoral condyle harvested 4 weeks post-ACLT of the left knee that was transfected with beta-galactosidase. Arrows point to condylar regions showing obvious surface roughness and apparent fibrillations, demonstrating the induction of OA by the surgical procedure, and showing that beta-galactosidase expression does not rescue the pathology.

FIG. 8A shows femoral condyles from 4 week ACLT left knees transfected with Kras-2b. The condyle has the high-surface reflectance typical of normal cartilage and there are no apparent regions of fibrillations present. These results were typical of the Kras-2b specimens. FIG. 8B shows femoral condyles from 4 week ACLT left knees treated with a high dose of Kras-2b, showing typically ‘normal’ macroscopic cartilage surface. Dose levels (high vs. low) were ascribed retrospectively from the real-time RT-PCR assays for Kras expression shown in FIG. 2.

FIG. 9 shows sections of control, β-galactosidase, (top panel) and Kras-2b (bottom panel) transfected knees. The sections show dramatic reduction in cartilage height, safranin-o staining and surface roughness. Safranin-O specifically stains GAGs (Glucosamino-glycans) in the cartilage. The reduction in Safranin-o staining reflects loss of GAGs and other proteoglycans that are indicators of cartilage health.

FIG. 10 shows sections of β-galactosidase transfected knees, demonstrating prominent lesions in the cartilage surface 4 weeks post-ACLT.

FIG. 11 shows sections of Kras-2b transfected 4-week ACLT knees. The knees showed nearly complete protection of the cartilage from degradation. Additionally, in the Kras-2b transfected knees the—cartilage height was preserved, the proteoglycans were preserved (safranin-O stained red) with minor loss of proteoglycans in the medial condyle (left panel). The chondrocytes maintained their columnar alignment. Most significantly, by 12 weeks post-post ACLT the cartilage surface was completely intact.

FIGS. 12A-F shows sections of β-galactosidase and Kras-2b tibial plateaus. The sections show dramatic differences in cartilage preservation between β-galactosidase transfected knees and Kras-2b transfected knees. The proteoglycans are preserved (Safranin-O: red staining) and the cartilage height and surface is maintained in the Kras-2b transfected knees.

### SUMMARY OF THE INVENTION

The present invention relates to methods and compositions for introducing exogenous nucleic acids into the joint of a mammal, thereby treating and/or preventing a joint disease or disorder.

Various aspects of the present invention include the following:

In one aspect, the invention comprises compositions and methods for introducing an exogenous nucleic acid encoding Kras-2b or an effector thereof into a target cell in the joint of a mammal. The composition can comprise a synthetic liposome comprising an exogenous nucleic acid encoding Kras-2b or an effector thereof, at least one ligand, and a poly-1-lysine polymeric scaffold, wherein said polymeric scaffold is attached to both said ligand and said nucleic acid. The liposome is selected from the group consisting of cationic liposomes, anionic liposomes, and synthetic lipid microspheres. The ligand can be any ligand, and in one aspect, the ligand is transferrin. The polymeric scaffold is selected from the group consisting of positively charged polymeric scaffolds, uncharged polymeric scaffolds, homopolymeric scaffolds, and poly-L-lysine scaffolds. In one aspect, the nucleic acid is contained within a vector, which can be a viral or non-viral vector. In a further aspect, the gene encoding Kras-2b is operably linked to a heterologous promoter selected from the group consisting of a heterologous constitutive promoter and a heterologous inducible promoter.

In another aspect, prior to introduction of the vector, the target cells and/or tissues are treated with a permeabilization agent selected from the group consisting of lysolecithin, polyoxyethylenesorbitan monolaurate, octylphenoxy polyethoxy ethanol, t-octylphenoxypolyethoxyethanol, phosphatidylcholine, phospholipase, streptolysins, equinatoxins, methyl-beta-cyclodextrin, sodium caprate, decanoylcarnitine, tartaric acid, alamethicin, Saponin, non-ionic detergents (NPs), Tween(s), ultrasonic shock, osmotic shock, and electric pulses.

The invention further contemplates a method of protecting a joint from degradation in a mammal, comprising delivering a vector to target cells in the joint of said mammal, the vector comprising a gene encoding Kras-2b or an effector thereof operably linked to a promoter. In one aspect, the vector is introduced intra-synovially, intramuscularly, subcutaneously and/or transdermally. Exemplary Kras-2b sequences are provided herein at Table I, and are described in more detail below. In one aspect, the gene encoding Kras-2b is a variant of a wild-type Kras-2b gene. In another aspect, the effector of Kras-2b is any molecule that modulates a Kras-2b signaling pathway, which includes but is not limited to, Kras-2a, KSR, MP1, N-Ras, H-Ras, Raf, dominant negative mutants of ras or Kras, truncations of ras and Raf, RAP, RAC, PAC, 14-3-3, MAPK, MAPKK (or variants), MAPKKK (or variants), MEK½, ERK 1/2, mTOR, TOR, Tob1, P38, PI3K, Ink4, Akt, JNK, S6, S6 kinases, cyclins, and Smads and other members of the ras signal transduction cascade.

It is contemplated that the target cell is a mammalian cell, including but not limited to a mouse cell, rat cell, rabbit cell, dog cell, cat cell, cow cell, goat cell, sheep cell, pig cell, horse cell, non-human primate cell, or a human cell. In one aspect the target cell is a human cell. Further, the target cell can be selected from the group consisting of primary perichondrial cells, cartilage cells, flexor tendon cells, tendon sheath cells, tendon pulley cells, epithelial skin cells, skin, muscle, fat, fibroblasts, tracheal cells, esophageal cells, dendritic cells, hair cells, keratinocytes, cells in the hair shaft, nerve cells, vascular smooth muscle cells, blood vessel walls, endothelial cells, immune cells (B-cells, T-cells, Neutrophils, NTK cells, macrophages, mononuclear cells, osteoblasts, osteoclasts, liver cells, heart smooth muscles, bronchial aveolii, other pulmonary cells, buccal cavity cells, nasal cavity cells, mononuclear marrow cells, multinucleated marrow cells, spinal chord, motoneurons, ganglia, and cells of the CNS.

The methods provided herein are useful for preventing and/or treating osteoarthritis. In another aspect, the method treats and/or prevents joint diseases and disorders related to arthritis and inflammation. In other aspects, these methods are useful for treating or preventing Rheumatoid arthritis (RA), Crystal disease, Coeliac disease, diabetes mellitus type 1 (IDDM), systemic lupus erythematosus (SLE), Sjögren's syndrome, multiple sclerosis (MS), Hashimoto's thyroiditis, Graves' disease and idiopathic thrombocytopenic purpura. In certain aspects, the Kras-2b molecules, effectors thereof and modulatory compounds treat or prevent the joint disease or disorder by decreasing the cellular proliferation index of one or more tissues of the joint. The decrease in the cellular proliferation index can be more than 10%, 20%, 30%, 40%, 50%, 60%, 70%, 80%, 90%, 95%, 99%, or more.

The invention further contemplates a method of expressing Kras-2b in a cell of the joint of a mammal in vivo, comprising (a) providing a vector encoding Kras-2b; (b) introducing the vector into a target cell in the joint of a mammal in vivo; and (c) maintaining the cell in vivo under conditions permitting expression of Kras-2b in the cell. It also encompasses a method of treating a mammal with osteoarthritis, comprising introducing into the patient an expression vector encoding Kras-2b, such that an amount of Kras-2b effective to alleviate one or more symptoms of osteoarthritis is expressed in the mammal. The compositions of the current invention are administered according to methods well known in the art. They may be administered once, or more than once. If administered two or more times, the timing of the administrations may be varied. For example, the compositions may be administered on a daily, weekly, biweekly, monthly, or variable schedule.

The invention further contemplates a device and kits containing a pharmaceutical composition comprising a therapeutic agent which is (i) one or more Kras-2b polypeptides or an effector thereof, (ii) one or more polynucleotides capable of expressing one or more Kras-2b polypeptides or an effector thereof in a target cell in the joint of a mammal, said device and kits being suitable for intra-synovial delivery of said composition.

### DETAILED DESCRIPTION OF PREFERRED EMBODIMENTS

The present invention relates to methods and compositions for introducing exogenous nucleic acids into the joint of a mammal, thereby treating and/or preventing a joint disease or disorder.

In one aspect, the invention comprises compositions and methods for introducing an exogenous nucleic acid encoding Kras-2b or an effector thereof into a target cell in the joint of a mammal. The composition can comprise a synthetic liposome comprising an exogenous nucleic acid encoding Kras-2b or an effector thereof, at least one ligand, and a poly-1-lysine polymeric scaffold, wherein said polymeric scaffold is attached to both said ligand and said nucleic acid. The liposome is selected from the group consisting of cationic liposomes, anionic liposomes, and synthetic lipid microspheres. The ligand can be any ligand, and in one aspect, the ligand is transferrin. The polymeric scaffold is selected from the group consisting of positively charged polymeric scaffolds, uncharged polymeric scaffolds, homopolymeric scaffolds, and poly-L-lysine scaffolds. In one aspect, the nucleic acid is contained within a vector, which can be a viral or non-viral vector. In a further aspect, the gene encoding Kras-2b is operably linked to a heterologous promoter selected from the group consisting of a heterologous constitutive promoter and a heterologous inducible promoter.

In another aspect, prior to introduction of the vector, the target cells are treated with a permeabilization agent selected from the group consisting of lysolecithin, polyoxyethylenesorbitan monolaurate, octylphenoxy polyethoxy ethanol, t-octylphenoxypolyethoxyethanol, phosphatidylcholine, phospholipase, streptolysins, equinatoxins, Methyl-beta-cyclodextrin, sodium caprate, decanoylcarnitine, tartaric acid, alamethicin, Saponin, non-ionic detergents (NPs), Tween(s), ultrasonic shock, osmotic shock, and electric pulses.

The invention further contemplates a method of protecting a joint from degradation in a mammal, comprising delivering a vector to target cells in the joint of said mammal, the vector comprising a gene encoding Kras-2b or an effector thereof operably linked to a promoter. In one aspect, the vector is introduced intra-synovially, intramuscularly, subcutaneously and/or transdermally. Exemplary Kras-2b sequences are provided herein at Table I, and are described in more detail below. In one aspect, the gene encoding Kras-2b is a variant of a wild-type Kras-2b gene. In another aspect, the effector of Kras-2b is any molecule that modulates a Kras-2b signaling pathway, which includes but is not limited to, Kras-2a, KSR, MP1, N-Ras, H-Ras, Raf, dominant negative mutants of ras or Kras, truncations of ras and Raf, RAP, RAC, PAC, 14-3-3, MAPK, MAPKK (or variants), MAPKKK (or variants), MEK½, ERK 1/2, mTOR, TOR, Tob1, P38, PI3K, Ink4, Akt, JNK, S6, S6 kinases, cyclins, and Smads and other members of the ras signal transduction cascade.

It is contemplated that the target cell is a mammalian cell, including but not limited to a mouse cell, rat cell, rabbit cell, dog cell, cat cell, cow cell, goat cell, sheep cell, pig cell, horse cell, non-human primate cell, or a human cell. In one aspect the target cell is a human cell. Further, the target cell can be selected from the group consisting of primary perichondrial cells, cartilage cells, flexor tendon cells, tendon sheath cells, tendon pulley cells, epithelial skin cells, skin, muscle, fat, fibroblasts, dendritic cells, hair cells, keratinocytes, cells in the hair shaft, nerve cells, vascular smooth muscle cells, blood vessel walls, immune cells (B-cells, T-cells, Neutrophils, NTK cells, macrophages, mononuclear cells, osteoblasts, osteoclasts, liver cells, heart smooth muscles, bronchial aveolii, other pulmonary cells, buccal cavity cells, nasal cavity cells, mononuclear marrow cells, multinucleated marrow cells, spinal chord, motoneurons, ganglia, and cells of the CNS.

The methods provided herein are useful for preventing and/or treating osteoarthritis. In another aspect, the method treats and/or prevents joint diseases and disorders related to arthritis and inflammation. In other aspects, these methods are useful for treating or preventing Rheumatoid arthritis (RA), Crystal disease, Coeliac disease, diabetes mellitus type 1 (IDDM), systemic lupus erythematosus (SLE), Sjögren's syndrome, multiple sclerosis (MS), Hashimoto's thyroiditis, Graves' disease and idiopathic thrombocytopenic purpura. In certain aspects, the Kras-2b molecules, effectors thereof and modulatory compounds treat or prevent the joint disease or disorder by decreasing the cellular proliferation index of one or more tissues of the joint. The decrease in the cellular proliferation index can be more than 10%, 20%, 30%, 40%, 50%, 60%, 70%, 80%, 90%, 95%, 99%, or more.

The invention further contemplates a method of expressing Kras-2b in a cell of the joint of a mammal in vivo, comprising (a) providing a vector encoding Kras-2b; (b) introducing the vector into a target cell in the joint of a mammal in vivo; and (c) maintaining the cell in vivo under conditions permitting expression of Kras-2b in the cell. It also encompasses a method of treating a mammal with osteoarthritis, comprising introducing into the patient an expression vector encoding Kras-2b, such that an amount of Kras-2b effective to alleviate one or more symptoms of osteoarthritis is expressed in the mammal. The compositions of the current invention are administered according to methods well known in the art. They may be administered once, or more than once. If administered two or more times, the timing of the administrations may be varied. For example, the compositions may be administered on a daily, weekly, biweekly, monthly, or variable schedule.

The invention further contemplates a device and kits containing a pharmaceutical composition comprising a therapeutic agent which is (i) one or more Kras-2b polypeptides or an effector thereof, (ii) one or more polynucleotides capable of expressing one or more Kras-2b polypeptides or an effector thereof in a target cell in the joint of a mammal, said device and kits being suitable for intra-synovial delivery of said composition.

### DEFINITIONS

A “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides.

The terms “nucleic acid” or “nucleic acid sequence” refers to oligonucleotides, nucleotides, polynucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin. The term encompasses nucleic acids, i.e., oligonucleotides, containing known analogues of natural nucleotides, naturally occurring nucleic acids, synthetic nucleic acids, and recombinant nucleic acids. The term also encompass ses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

The nucleic acids used to practice this invention, whether RNA, iRNA, siRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Alternatively, nucleic acids can be obtained from commercial sources.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In practicing the invention, nucleic acids of the invention or modified nucleic acids of the invention, can be reproduced by amplification. Amplification can also be used to clone or modify the nucleic acids of the invention. Thus, the invention provides amplification primer sequence pairs for amplifying nucleic acids of the invention. One of skill in the art can design amplification primer sequence pairs for any part of or the full length of these sequences.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; and Sooknanan (1995) Biotechnology 13:563-564.

“Recombinant,” as applied to a polynucleotide, means that the polynucleotide is the product of various combinations of cloning, restriction and/or ligation steps, and other procedures that result in a construct that is distinct from a polynucleotide found in nature.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters. The term “gene” includes a nucleic acid sequence comprising a segment of DNA involved in producing a transcription product (e.g., a message), which in turn is translated to produce a polypeptide chain, or regulates gene transcription, reproduction or stability. Genes can include regions preceding and following the coding region, such as leader and trailer, promoters and enhancers, as well as, where applicable, intervening sequences (introns) between individual coding segments (exons).

The term “genome” refers to the complete genetic material of an organism.

In one aspect, the term “transformation” refers to the transfer of a nucleic acid sequence into the genome of a host cell, resulting in genetically stable inheritance. A “host cell” is a cell that has been transformed, or is capable of transformation, by an exogenous nucleic acid molecule. Host cells containing the transformed nucleic acid sequences are referred to as “transgenic” cells, and organisms comprising transgenic cells are referred to as “transgenic organisms”. The terms “transformed”, “transduced”, “transgenic”, and “recombinant” refer to a host cell or organism into which a heterologous nucleic acid molecule has been introduced. The nucleic acid molecule can be stably integrated into the genome generally known in the art and are disclosed in Sambrook and Russell, infra. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

The terms “polypeptide,” “peptide,” and “protein” are used interchangeably to refer to polymers of amino acids of any length. These terms also include proteins that are post-translationally modified through reactions that include glycosylation, acetylation and phosphorylation.

Peptides and polypeptides include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered from an exemplary polypeptide of the invention. In one aspect, a mimetic composition is used in a composition, cell system or process of the invention (e.g., a host cell having a plasmid expressing at least one enzyme of the invention).

Polypeptide mimetic compositions of the invention can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH2— for —C(═O)—NH—), aminomethylene (CH2—NH), ethylene, olefin (CH═CH), ether (CH2—O), thioether (CH2—S), tetrazole (CN4—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY). A residue, e.g., an amino acid, of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R- or S-form.

Polypeptides used to practice the method of the invention (e.g., beta-galactosidase, Kras-2b) can be modified by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

The term “exogenous,” when used in reference to a nucleic acid molecule, means that the nucleic acid molecule is from a source other than the target cell, into which the nucleic acid molecule is to be introduced. It should be recognized, however, that the exogenous nucleic acid molecule can be from other cells from the same type of organism as the target cells.

A “promoter,” as used herein, refers to a polynucleotide sequence that controls or modulates transcription of a gene or nucleic acid sequence to which it is operably linked. A large number of promoters, including constitutive, inducible and repressible promoters, from a variety of different sources, are well known in the art and are available as or within cloned polynucleotide sequences (from, e.g., depositories such as the ATCC as well as other commercial or individual sources).

Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

“Operably linked” refers to a juxtaposition, wherein the components so described are in a relationship permitting them to function in their intended manner. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. In other aspects, enhancers need not be physically contiguous or located in close proximity to the coding sequences whose transcription they enhance. A polyadenylation sequence is operably linked to a coding sequence if it is located at the downstream end of the coding sequence such that transcription proceeds through the coding sequence into the polyadenylation sequence.

A “replicon” refers to a polynucleotide comprising an origin of replication which allows for replication of the polynucleotide in an appropriate host cell. Examples include replicons of a target cell into which a heterologous nucleic acid might be integrated (e.g., nuclear and mitochondrial chromosomes), as well as extrachromosomal replicons (such as replicating plasmids and episomes).

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available (see, e.g., the various references cited below).

A “recombinant viral vector” refers to a viral vector comprising one or more heterologous genes or sequences. Since many viral vectors exhibit size-constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation) (see, e.g., the references and illustrations below). Modified viral vectors in which a polynucleotide to be delivered is carried on the outside of the viral particle have also been described (see, e.g., Curiel, D T, et al. PNAS 88: 8850-8854, 1991).

Viral “packaging” as used herein refers to a series of intracellular events that result in the synthesis and assembly of a viral vector. Packaging typically involves the replication of the “pro-viral genome”, or a recombinant pro-vector typically referred to as a “vector plasmid” (which is a recombinant polynucleotide than can be packaged in an manner analogous to a viral genome, typically as a result of being flanked by appropriate viral “packaging sequences”), followed by encapsidation or other coating of the nucleic acid. Thus, when a suitable vector plasmid is introduced into a packaging cell line under appropriate conditions, it can be replicated and assembled into a viral particle. Viral “rep” and “cap” genes, found in many viral genomes, are genes encoding replication and encapsidation proteins, respectively. A “replication-defective” or “replication-incompetent” viral vector refers to a viral vector in which one or more functions necessary for replication and/or packaging are missing or altered, rendering the viral vector incapable of initiating viral replication following uptake by a host cell. To produce stocks of such replication-defective viral vectors, the virus or pro-viral nucleic acid can be introduced into a “packaging cell line” that has been modified to contain genes encoding the missing functions which can be supplied in trans). For example, such packaging genes can be stably integrated into a replicon of the packaging cell line or they can be introduced by transfection with a “packaging plasmid” or helper virus carrying genes encoding the missing functions.

The term “overexpression” refers to the level of expression in transgenic cells or organisms that exceeds levels of expression in normal or untransformed cells or organisms.

A “detectable marker gene” is a gene that allows cells carrying the gene to be specifically detected (e.g., distinguished from cells which do not carry the marker gene). A large variety of such marker genes are known in the art. Examples thereof include detectable marker genes which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining, as described below.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., WO 92/08796, published 29 May 1992, and WO 94/28143, published 8 Dec. 1994). Such marker genes can provide an added measure of control that can be advantageous in gene therapy contexts.

By “subject” is meant an organism, which is a donor or recipient of explanted cells or the cells or tissues themselves. “Subject” also refers to an organism (including a mammal or a human) to which the nucleic acid molecules of the invention can be administered. In one embodiment, a subject is a mammal or mammalian cell. The term “mammal” refers to any organism classified as a mammal, including mice, rats, rabbits, dogs, cats, cows, goats, sheep, pigs, horses, monkeys, and humans. In one embodiment of the invention, the mammal is a rabbit. In another embodiment of the invention, the mammal is a human. In another embodiment, a subject is a human subject or human cell.

“Treatment” or “therapy” as used herein refers to administering, to a mammal, agents that are capable of eliciting a prophylactic, curative or other beneficial effect in the individual.

“Gene therapy” as used herein refers to administering, to a mammal, vectors comprising a therapeutic gene.

A “therapeutic polynucleotide” or “therapeutic gene” refers to a nucleotide sequence that is capable, when transferred to a mammal, of eliciting a prophylactic, curative or other beneficial effect in the mammal.

As used herein, the singular form “a”, “an”, and “the” includes plural references unless indicated otherwise. For example, “a” target cell includes one or more target cells.

### REFERENCES

The practice of the present invention will employ, unless otherwise indicated, conventional techniques of molecular biology and the like, which are within the skill of the art. Such techniques are explained fully in the literature. See e.g., Molecular Cloning: A Laboratory Manual, (J. Sambrook et al., Cold Spring Harbor Laboratory, Cold Spring Harbor, N.Y., 1989); Current Protocols in Molecular Biology (F. Ausubel et al. eds., 1987 and updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al. eds., Bartlett Publ. 1990); Gene Transfer and Expression (M. Kriegler, Stockton Press 1990); Recombinant DNA Methodology (R. Wu et al. eds., Academic Press 1989); PCR: A Practical Approach (M. McPherson et al., IRL Press at Oxford University Press 1991); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells, 2nd Ed. (R. Freshney et al. eds., Alan R. Liss 1987); Flow Cytometry and Sorting (M. Melamed et al. eds., Wiley-Liss 1990); the series Methods in Enzymology (Academic Press, Inc.); Techniques in Immunocytochemistry, (G. Bullock & P. Petrusz eds., Academic Press 1982, 1983, 1985, 1989); Handbook of Experimental Immunology, (D. Weir & C. Blackwell, eds.); Cellular and Molecular Immunology (A. Abbas et al., W.B. Saunders Co. 1991, 1994); Current Protocols in Immunology (J. Coligan et al. eds. 1991); the series Annual Review of Immunology; the series Advances in Immunology; Oligonucleotide Synthesis (M. Gait ed., 1984); and Animal Cell Culture (R. Freshney ed., IRL Press 1987).

### INCORPORATION BY REFERENCE

References cited within this application, including GenBank accession numbers, patents, published patent applications and other publications, are hereby incorporated by reference.

### DESCRIPTION OF VARIOUS EMBODIMENTS OF THE INVENTION

Various aspects of the present invention are summarized below and further described and illustrated in the subsequent detailed descriptions and examples.

Kras-2b Peptides

The term Kras-2b as used herein will be understood to refer to any form or analog of Kras-2b peptide that retains the biological activity of native Kras-2b and preferably retains the activity of Kras-2b that is expressed during embryonic development. Kras knock-out mice, unlike those lacking the other ras homologues such as N- or H-ras, were embryonic lethal.

The Kras-2b used in the methods and compositions of the present invention may be identical to or comprise the sequences shown in Table I, and identified by accession number. Kras-2b also refers to variant polypeptides having an amino acid sequence which varies from those identified in Table I. Alternatively, Kras-2b may be chemically-modified.

TABLE I

| ACCESSION # | DESCRIPTION | SPECIES |
|---|---|---|
| NC_006479.2 | v-Ki-ras2 Kirsten rat sarcoma viral oncogene homolog | Pan |
| | Annotation: Chromosome 12, NC_006479.2 | troglodytes |
| | (63755647 . . . 63802210) | |
| | GeneID: 473387 | |
| NC_000072.5 | v-Ki-ras2 Kirsten rat sarcoma viral oncogene homolog | Mus |
| | Other Aliases: AI929937, K-ras, Ki-ras, Kras-2, Kras2, | musculus |
| | MGC7141, p21B, ras | |
| | Other Designations: Kirsten rat sarcoma oncogene 2, | |
| | expressed; c-K-ras2 protein; p21 protein | |
| | Chromosome 6, (145165227 . . . 145198751, complement) | |
| | GeneID: 16653 | |
| NC_007861.1 | similar to GTPase KRas (K-Ras 2) (Ki-Ras) (c-K-ras) (c-Ki-ras) | Macaca |
| | Chromosome 4, (54450447 . . . 54452414) | mulatta |
| | GeneID: 714730 | |
| NC_000012.10 | Official Symbol KRAS and Name: v-Ki-ras2 Kirsten rat | Homo |
| | sarcoma viral oncogene homolog Other Aliases: C-K-RAS, | sapiens |
| | K-RAS2A, K-RAS2B, K-RAS4A, K-RAS4B, KI-RAS, | |
| | KRAS1, KRAS2, NS3, RASK2 | |
| | Other Designations: K-ras p21 protein; Kirsten rat sarcoma-2 | |
| | viral (v-Ki-ras2) oncogene homolog; PR310 c-K-ras oncogene; | |
| | c-K-ras2 protein; c-Kirsten-ras protein; cellular c-Ki-ras2 | |
| | proto-oncogene; oncogene KRAS2; transforming protein p21; | |
| | v-Ki-ras2 Kirsten rat sarcoma 2 viral oncogene homolog | |
| | Chromosome: 12; Location: 12p12.1 | |
| | Annotation: Chromosome 12, (25249447 . . . 25295121, | |
| | complement) | |
| | MIM: 190070 | |
| | GeneID: 3845 | |
| NC_007868.1 | similar to GTPase KRas (K-Ras 2) (Ki-Ras) (c-K-ras) (c-Ki-ras) | Macaca |
| | Chromosome: 11 | mulatta |
| | Annotation: Chromosome 11, GeneID: 707977 | |
| BT007153.1 | v-Ki-ras2 Kirsten rat sarcoma 2 viral oncogene homolog | Homo |
| | mRNA, complete cds | sapiens |
| NM_004985.3 | Homo sapiens v-Ki-ras2 Kirsten rat sarcoma viral oncogene | Homo |
| | homolog (KRAS), transcript variant b, mRNA | sapiens |
| CR625034.1 | full-length cDNA clone CS0DI073YJ08 of Placenta Cot | Homo |
| | 25-normalized of Homo sapiens (human) | sapiens |
| BC010502.1 | Homo sapiens v-Ki-ras2 Kirsten rat sarcoma viral oncogene | Homo |
| | homolog, mRNA (cDNA clone | sapiens |
| | IMAGE:3450509), complete cds | |
| BC013572.2 | Homo sapiens v-Ki-ras2 Kirsten rat sarcoma viral oncogene | Homo |
| | homolog, mRNA (cDNA clone MGC:8977 | sapiens |
| | IMAGE:3878884), complete cds | |
| NM_033360.2 | Homo sapiens v-Ki-ras2 Kirsten rat sarcoma viral oncogene | Homo |
| | homolog (KRAS), transcript variant a, mRNA | sapiens |
| AF493917.1 | Homo sapiens Ras family small GTP binding protein K-Ras2 | Homo |
| | (KRAS) mRNA, complete cds | sapiens |
| BC029545.1 | Homo sapiens v-Ki-ras2 Kirsten rat sarcoma viral oncogene | Homo |
| | homolog, mRNA (cDNA clone IMAGE:5301134) | sapiens |
| M17087.1 | Human c-ras-Ki-2 activated oncogene mRNA, partial cds | Homo |
| | | sapiens |
| M35505.1 | Human PR371 c-K-ras oncogene mRNA, 5′ end | Homo |
| | | sapiens |
| M35504.1 | Human PR310 c-K-ras protein mRNA, 5′ end | Homo |
| | | sapiens |
| NP_203524.1 | c-K-ras2 protein isoform a | H. sapiens |
| XP_416436.1 | PREDICTED: similar to turkey K-Ras | G. gallus |
| NP_067259.3 | c-K-ras2 protein | M. musculus |
| NP_113703.1 | c-K-ras2 protein | R. norvegicus |
| NP_001084379.1 | proto-oncogene K-Ras2A GeneID: 373672 | X. laevis |
| NP_001003744.1 | hypothetical protein LOC445289 | D. rerio |
| NP_476699.1 | CG9375-PA | D. melanogaster |
| NP_502213.3 | LEThal family member (let-60) | C. elegans |
| NP_014301.1 | Ras proto-oncogene homolog. Ras2 is involved in growth on | S. cerevisiae |
| | non-fermentable carbon sources, the starvation response, | |
| | sporulation, pseudohyphal growth and aging.; Ras2p | |
| NP_593579.1 | hypothetical protein SPAC17H9.09c | S. pombe |
| XP_322918.1 | RAS-2 PROTEIN | N. crassa |
| GeneID: 281887 | Kirsten rat sarcoma viral (Kras-2) oncogene homolog | B. Taurus |
| NC_000012.10 | sarcospan (Kras oncogene-associated gene) Chromosome: 12; | H. sapiens |
| | Location: 12p11.2 | |
| NC_008808.1 | v-Ki-ras2 Kirsten rat sarcoma viral oncogene homolog | Monodelphis |
| | [GeneID: 554186]; K-ras proto-oncogene; p21-ras | domestica |

A variant of Kras-2b may be a naturally occurring variant, for example a variant which is expressed by a non-human species. Also, variants of Kras-2b include sequences which vary from the disclosed sequences but are not necessarily naturally occurring. Over the entire length of the naturally occurring amino acid sequence, a variant will preferably be at least 80% homologous to that sequence based on amino acid identity. More preferably, the polypeptide is at least 85% or 90% and more preferably at least 95%, 97% or 99% homologous based on amino acid identity to the naturally occurring amino acid sequence over the entire sequence. There may be at least 80%, for example at least 85%, 90% or 95%, amino acid identity over a stretch of 40 or more, for example 60, 80, 100, 120, 140 or 160 or more, contiguous amino acids (“hard homology”).

Homology may be determined using any method known in the art. Homology or sequence identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence, e.g., a sequence of the invention, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Amino acid substitutions may be made to the amino acid sequence of SEQ ID NO:2, for example from 1, 2, 3, 4 or 5 to 10, 20 or 30 substitutions. Conservative substitutions may be made. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a polypeptide, resulting in modification of the structure of the polypeptide without significantly altering its biological activity.

One or more amino acid residues of the amino acid sequence of SEQ ID NO:2 may alternatively or additionally be deleted. 1, 2, 3, 4, 5, 6, 7, 8, 9, 10, 20 or 30 or more residues may be deleted.

Kras-2b also includes fragments of the above-mentioned sequences. Such fragments retain Kras-2b activity. Fragments may be at least from 120 or 140 amino acids in length. Such fragments may be used to produce chimeric agents as described in more detail below.

Kras-2b includes chimeric proteins comprising fragments or portions of the endogenous sequences described in Table I or variants or homologs thereof. One or more amino acids may be alternatively or additionally added to the polypeptides described above. An extension may be provided at the N-terminus or C-terminus of the amino acid sequence of the naturally occurring amino acid sequence or polypeptide variant or fragment thereof. The extension may be quite short, for example from 1 to 10 amino acids in length. Alternatively, the extension may be longer. A carrier protein may be fused to an amino acid sequence described above. A fusion protein incorporating one of the polypeptides described above can thus be used in the invention.

Kras-2b also includes naturally occurring amino acid sequence or variants or homologs thereof that have been chemically-modified. A number of side chain modifications are known in the art and may be made to the side chains of the proteins or peptides discussed above. Such modifications include, for example, glycosylation, phosphorylation, modifications of amino acids by reductive alkylation by reaction with an aldehyde followed by reduction with NaBH₄, amidination with methylacetimidate or acylation with acetic anhydride. The modification is preferably glycosylation.

The Kras-2b polypeptide may be made synthetically or by recombinant means using methods known in the art. The amino acid sequence of proteins and polypeptides may be modified to include non-naturally occurring amino acids or to increase the stability of the compound. When the proteins or peptides are produced by synthetic means, such amino acids may be introduced during production. The proteins or peptides may also be modified following either synthetic or recombinant production.

Kras-2b may also be produced using D-amino acids. In such cases the amino acids will be linked in reverse sequence in the C to N orientation. This is conventional in the art for producing such proteins or peptides.

Kras-2b may be produced in a cell by in situ expression of the polypeptide from a recombinant expression vector. The expression vector optionally carries an inducible promoter to control the expression of the polypeptide. Kras-2b or analog thereof may be produced in large scale following purification by any protein liquid chromatography system after recombinant expression. Preferred protein liquid chromatography systems include FPLC, AKTA systems, the Bio-Cad system, the Bio-Rad BioLogic system and the Gilson HPLC system.

Commercially available forms of Kras-2b or analogs thereof may be used in the invention.

The biological activity of “fragments” or “variants” of a Kras-2b polypeptide may be determined by, for example, measuring RFLP analysis as described previously (James et al., (2003) Mol. Cancer. Res., 1:820-825; Schimanski et al., (1999) Cancer Research, 59:5169-5175.)

The term “substantially the same or more activity” includes “fragments” or “variants” of an Kras-2b polypeptide having at least 50%, 60%, 70%, 80%, 90%, 95%, 100% or more of the biological activity of the endogenous Kras-2b polypeptide.

Kras-2b Polynucleotides

The invention may also involve using a polynucleotide which is capable of expressing Kras-2b in mammalian joints and/or in cartilage cells. Such a polynucleotide may be in the form of a vector capable of directing expression of Kras-2b in the perichondium. The resulting Kras-2b may then have a therapeutic effect (“gene therapy”). The polynucleotide may encode any of the forms of Kras-2b discussed above including the variants, fragments and chimeric proteins thereof.

The polynucleotide encoding Kras-2b may comprise a human sequence or a naturally occurring sequence variant, for example a variant which is expressed by a non-human species. Also, a polynucleotide encoding Kras-2b includes sequences which vary from the naturally occurring sequences but are not necessarily naturally occurring. Over the entire length of the amino acid sequence of the naturally occurring sequences disclosed in Table I, a variant will preferably be at least 80% homologous to that sequence based on nucleotide identity. More preferably, the polynucleotide is at least 85% or 90% and more preferably at least 95%, 97% or 99% homologous based on nucleotide identity to the nucleotide of the naturally occurring sequences disclosed in Table I over the entire sequence. There may be at least 80%, for example at least 85%, 90% or 95%, nucleotide identity over a stretch of 40 or more, for example 60, 80, 100, 120, 140 or 160 or more, contiguous nucleotides (“hard homology”). Homology may be determined as discussed above.

The polynucleotides may comprise DNA or RNA. They polynucleotides may include synthetic or modified nucleotides. A number of different types of modification to polynucleotides are known in the art. These include methylphosphate and phosphorothioate backbones, addition of acridine or polylysine chains at the 3′ and/or 5′ ends of the molecule. For the purposes of the present invention, it is to be understood that the polynucleotides described herein may be modified by any method available in the art.

Polynucleotides such as a DNA polynucleotide may be produced recombinantly, synthetically, or by any means available to those of skill in the art. They may also be cloned by standard techniques. The polynucleotides are typically provided in isolated and/or purified form.

Polynucleotides will generally be produced using recombinant means, for example using PCR (polymerase chain reaction) cloning techniques. This will involve making a pair of primers (e.g. of about 15-30 nucleotides) to a region of the required gene which it is desired to clone, bringing the primers into contact with DNA obtained from a suitable cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Although in general the techniques mentioned herein are well known in the art, reference may be made in particular to Sambrook et al, 1989.

As hereinbefore indicated, preferably the polynucleotide is used in an expression vector wherein it is operably linked to a control sequence which is capable of providing for the expression of the coding sequence in the joint of a mammal, such as in the perichondrium.

Therapeutic Applications

Administration of Kras-2b protein, a Kras-2b polynucleotide, an effector of has, or a modulatory compound (hereinafter a “therapeutic agent”) as discussed herein may be either for preventative or therapeutic purpose. When provided preventatively, the therapeutic agent is provided in advance of any symptoms. The preventative administration of the therapeutic agent serves to prevent or attenuate any symptoms. When provided therapeutically the therapeutic agent is provided at (or shortly after) the onset of a symptom of the joint disease or disorder. The therapeutic administration of the therapeutic agent serves to attenuate any actual exacerbation of the symptoms. The individual treated may be any mammal. In one aspect, the mammal is a human. In another aspect, the human has osteoarthritis or a degenerative joint disease. In other aspects, the joint diseases and disorders comprise arthritic pathologies and autoimmune diseases, including but not limited to Osteoarthritis (OA), Rheumatoid arthritis (RA), Crystal disease, Coeliac disease, diabetes mellitus type 1 (IDDM), systemic lupus erythematosus (SLE), Sjögren's syndrome, multiple sclerosis (MS), Hashimoto's thyroiditis, Graves' disease and idiopathic thrombocytopenic purpura.

The therapeutic agent may be administered in a medicament or pharmaceutical composition suitable for delivery to the joint. The pharmaceutical composition may also include a pharmaceutically acceptable excipient. Such an “excipient” generally refers to a substantially inert material that is nontoxic and does not interact with other components of the composition in a deleterious manner.

Pharmaceutically acceptable excipients include, but are not limited to, liquids such as water, saline, polyethyleneglycol, hyaluronic acid, glycerol and ethanol. Pharmaceutically acceptable salts can be included therein, for example, mineral acid salts such as hydrochlorides, hydrobromides, phosphates, sulphates, and the like; and the salts of organic acids such as acetates, propionates, malonates, benzoates, and the like.

It is also contemplated that a composition or medicament comprising the therapeutic agent can contain a pharmaceutically acceptable carrier that serves as a stabilizer, particularly for peptide, protein, polynucleotide or other like agents. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. It may also be useful to employ a charged lipid and/or detergent. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, for example, TWEEN® surfactants (Atlas Chemical Industries, Wilmington, Del.), polyoxyethylene ethers, for example Brij, pharmaceutically acceptable fatty acid esters, for example, lauryl sulfate and salts thereof (SDS), and like materials. A thorough discussion of pharmaceutically acceptable excipients, carriers, stabilizers and other auxiliary substances is available in Remingtons Pharmaceutical Sciences (Mack Pub. Co., N.J. 1991).

A composition comprising a prophylactically or therapeutically effective amount of the therapeutic agent described herein may conveniently be delivered to the joint of the mammal by intra-synovial injection. An appropriate effective amount may be determined by appropriate clinical testing and will vary with for example the activity of the therapeutic agent administered or nucleotide or protein levels induced. For example, a Kras-2b protein or a Kras-2b polynucleotide may be administered in microgram amounts. They are administered to the subject to be treated in a manner compatible with the dosage formulation, and in an amount that will be effective to bring about the desired effect. In one aspect, a Kras-2b polynucleotide is delivered, and the amount to be delivered may be 1 μg to 5 mg, for example 1 to 50 μg, depending on the subject to be treated. The exact amount necessary will vary depending on the age and general condition of the individual being treated and agent selected, as well as other factors. The Kras-2b protein or a Kras-2b polynucleotide may be administered once, or more than once. For example, it can be administered weekly, biweekly, monthly, or on an as-needed basis.

The Kras-2b protein or a Kras-2b polynucleotide may be administered on its own or in combination with another therapeutic compound. In particular, the Kras-2b protein or a Kras-2b polynucleotide may be administered in conjunction with a therapeutic compound used to treat osteoarthritis in the mammal. The Kras-2b protein or a Kras-2b polynucleotide and additional therapeutic compound may be formulated in the same or different compositions. In one embodiment, the Kras-2b protein or a Kras-2b polynucleotide is administered to an individual with osteoarthritis in combination with an additional therapeutic compound selected from the group consisting of an anti-inflammatory, a pain reliever, and a chondroprotective agent such as glucosamine and chrondroitin sulfate. The Kras-2b protein or a Kras-2b polynucleotide may be administered simultaneously, sequentially or separately from the additional therapeutic compound.

Thus, in a further aspect of the present invention there is provided a product for treatment of osteoarthritis comprising for simultaneous, separate or sequential administration (i) a first agent selected from (a) Kras-2b protein or a Kras-2b polynucleotide and (b) an additional therapeutic compound selected from the group consisting of an anti-inflammatory, a pain reliever, a chondroprotective agent such as glucosamine and chrondroitin sulfate. The two therapeutic compounds can be administered using the same or different routes. For example, the Kras-2b protein or a Kras-2b polynucleotide can be administered intra-synovially, while the other therapeutic compound such as the anti-inflammatory can be administered orally or parenterally. Preferably, such a product will provide for simultaneous, separate or sequential administration of Kras-2b protein or a Kras-2b polynucleotide and the other therapeutic compound.

A first agent as defined above and another therapeutic compound may, for example, be provided in the form of a single pharmaceutical composition suitable for delivery to the joint.

As described herein, a number of different vectors can be employed to deliver the Kras-2b transgene in vivo or ex vivo according to the present invention.

Compositions or products of the invention may conveniently be provided in the form of formulations suitable for administration into the joint (e.g. in the synovial space). A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences by E. W. Martin. See also Wang, Y. J. and Hanson, M. A. “Parental Formulations of Proteins and Peptides: Stability and Stabilizers,” Journals of Parental Sciences and Technology, Technical Report No. 10, Supp. 42:2 S (1988). In one aspect, vectors of the present invention are formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

For use by the physician, the compositions can be provided in dosage form containing an amount of a vector of the invention which will be effective in one or multiple doses to induce Kras-2b transgene delivery/expression at a desired level. As will be recognized by those in the field, an effective amount of therapeutic agent will vary with many factors including the age and weight of the patient, the patient's physical condition, and the level of enhancement of cardiac function desired, and other factors.

For nucleic acid vectors, the effective does of the compounds of this invention will typically be in the range of at least about 10⁷ nucleic acid particles, preferably about 10⁹ nucleic acid particles, and more preferably about 10¹¹ nucleic acid particles. The number of nucleic acid particles may, but preferably does not exceed 10¹⁴. As noted, the exact dose to be administered is determined by the attending clinician, but is preferably in 1 ml phosphate buffered saline.

The presently most preferred mode of administration in the case of degenerative joint disease is by intra-synovial injection.

Liposomal Transfection of Mammalian Perichondrial Cells

The present invention provides a composition for high efficiency receptor/liposome mediated delivery of nucleic acid molecules into cells. As disclosed herein, a composition of the invention is useful for introducing a nucleic acid molecule into a cell, ex vivo or in vivo, with transfection efficiencies of about 50% or greater. In particular, a composition of the invention is useful for introducing a nucleic acid molecule into a primary cell, for example, primary mammalian cells such as human cells. In one aspect, the primary mammalian cell is a cell in a joint, such as, but not limited to, perichondrial cells.

The term “transfection efficiency” refers to the percentage of target cells, within a population of target cells, which contain an introduced exogenous nucleic acid molecule. Transfection efficiency can be determined by transfecting a nucleic acid molecule encoding a reporter gene, for example, beta-galactosidase, into a population of target cells and determining the percentage of cells having beta-galactosidase activity. Thus, transfection efficiency can be determined by assaying for the gene product encoded by the introduced nucleic acid molecule. Reference herein to “high transfection efficiency” or the like refers to a transfection efficiency of at least about 50%, 55%, 60%, 65%, 70%, 75%, 80%, 85%, 90%, 91%, 92%, 93%, 94%, 95%, 96%, 97%, 98%, or 99%.

The present invention provides composition useful for introducing exogenous nucleic acid molecules, such as Kras-2b encoding nucleic acid sequences, into target cells with high efficiency. Certain compositions of the invention comprise a nucleic acid molecule, a cationic liposome, and a ligand that can bind to a cell surface receptor expressed by the target cells, wherein the ligand is attached to a positively charged polymeric scaffold.

As used herein, the term “introducing” when used in reference to an exogenous nucleic acid molecule, means that the nucleic acid molecule is delivered into a target cell; i.e., the nucleic acid molecule is transfected into the target cell. The term “target cell” is used herein to mean any cell into which an exogenous nucleic acid molecule is to be introduced. In particular, however, a target cell is characterized in that it expresses a particular cell surface receptor, which can bind the ligand component of a composition of the invention.

If desired, a nucleic acid molecule to be introduced into a target cell can be contained in a vector, which can be derived, for example, from a plasmid, bacteriophage or plant or animal virus. Such vectors can contain an origin of replication recognized by an appropriate host cell and, in the case of expression vectors, can contain a promoter or other regulatory region useful in a particular host cell or target cell. For example, a vector comprising the open reading frame of the active form of the beta-galactosidase gene ligated downstream of a human cytomegalovirus (CMV) promoter/enhancer sequence in a plasmid carrying the expression cassette for a gene for ampicillin resistance can be used to determine the transfection efficiency obtained using a composition and a method of the invention. One skilled in the art would know how to make and use or otherwise obtain other vectors (see, for example, Sambrook et al., Molecular Cloning: A Laboratory Manual (Cold Spring Harbor Press; Cold Spring Harbor, N.Y.; 1989), which is incorporated herein by reference).

Certain compositions of the present invention also comprise a liposome, including cationic, anionic and synthetic liposomes. As used herein, the term “liposome” refers to a vesicle bounded by a lipid bilayer. A “cationic liposome” has a net positive charge. Liposomes, including cationic liposomes, are well known in the art and can be prepared using routine methods (see, for example, Brant et al., Am. Fed. Med. Res. 45 (1):159A (1997), which is incorporated herein by reference; Feigner et al., supra (1987); and Wheeler et al., supra (1996)) or using commercially available kits such as DOTAP (Avanti Polar Lipids, Alabaster, Ala.); DOSPA (Life Technologies, Inc., Gaithersburg, Md.); and DDAB (Kodak, Rochester, N.Y.). Anionic liposomes and synthetic lipid microspheres can also be used in the present invention (see, for example, Lasic, American Scientist 80:20-31 (1992), which is incorporated herein by reference).

As used herein, the term “ligand” is used broadly herein to refer to a molecule that can bind to a receptor expressed on the surface of a target cell or, conversely, to a receptor that can bind a molecule expressed on the surface of a target cell. For example, the “ligand” can be transferrin, which can bind to a transferrin receptor expressed on the surface of a target cell such as a perichondrial cell. In other aspects, the “ligand” can be, for example, an anti-CD4 antibody, which binds to CD4 expressed on a target cell such as a T cell.

In one aspect of the invention, the ligand is attached to a positively charged polymeric scaffold by a covalent bond or other bond that is relatively stable under physiological conditions, including in vivo or in tissue culture. Thus, the ligand can be attached by noncovalent or ionic interaction between the positively charged polymeric scaffold and the ligand, provided the ligand is sufficiently anionic such that the interaction is maintained under physiological conditions. The use of a tether can facilitate binding and optimize the ratio of the scaffold to the ligand. A tether can be, for example, biotin, which is attached to the scaffold and binds avidin, which can be attached to the ligand.

A ligand useful in the invention can be any ligand that can bind to a receptor expressed on the surface of the target cell; or any receptor or other binding molecule that can bind to a molecule expressed on the surface of the target cell. For example, the ligand can be transferrin, which is covalently bound to poly-L-lysine. Other ligands useful in certain compositions of the invention include, for example, insulin, folate or cholera toxin (Cheng, (1996) Human Gene Ther., 7:275-282; Lee and Huang, J. Biol. Chem. 271 (14):8481-8487 (1996), which are incorporated herein by reference). In addition, other ligands that can be internalized upon binding to their receptors would be known to one in the art and would depend, for example, on the particular target cell to be transfected. Thus, the selection of the ligand will be based on the desired target cell.

An advantage of the present invention is the ease of preparation of the composition, since all of the components are commercially available. Reagents such as transferrin and poly-L-lysine, for example, can be purchased from Sigma Chemical Co. (St. Louis, Mo.). In addition, a composition of the invention poses only a low risk of generating a host immune response. For example, a ligand prepared from a particular animal source can be used for transfecting cells of that same animal species without concern of generating a host immune response against the ligand. There is also a minimal risk of immune response to the liposomes, as liposome toxicity studies have shown that they cause little or no host inflammatory or immune response (Stribling et al., Proc. Natl. Acad. Sci., USA 89:11277-11281 (1992); Alton et al., Nature Genet. 5:135-142 (1993)). Thus, a composition of the invention provides an advantage over the use of viral vectors, for example, in that the likelihood of an adverse immune response is minimized.

A composition of the invention contains a positively charged polymeric scaffold. As used herein, the term “polymeric scaffold” means a polymer having a net positive or neutral charge such that it can interact and form a complex with a negatively charged nucleic acid molecule. It should be understood that a polymeric scaffold can contain one or more units that have a negative charge, provided the polymer has a net positive or neutral charge.

A positively charged polymeric scaffold is exemplified herein by poly-L-lysine. Other scaffolds of the invention can be homo-polymeric amino acids, hetero-polymeric amino acids, amino acids not covalently bonded to each other, polymers containing positively charged moieties such as amine groups, poly-spermine or poly-spermidines. The term “homo-polymeric amino acids” means a covalently bonded polymer of the same amino acids either charged or uncharged and, which may interact with the nucleic acid molecule specifically or non-specifically. Conversely, the term “hetero-polymeric amino acids” means a covalently bonded polymer of different amino acids either charged or uncharged and, which may interact with the nucleic acid molecule specifically or non-specifically. Positively charged polymeric scaffolds with modifications or variations to their lengths are contemplated within the present invention, provided they allow high transfection efficiency as defined herein. For example, the molecular weight of poly-L-lysine can be from 15 kDa to 150 kDa. Modifications and or variations in the length of the positively charged polymeric scaffold can be made and the effect they have on transfection efficiency can be determined using methods as disclosed herein or methods otherwise known in the art.

In one aspect of the invention, a method of the invention is performed by permeabilizing the target cells and contacting the cells with a composition comprising an exogenous nucleic acid molecule, a cationic liposome and a ligand that binds to a receptor expressed on the surface of the target cells, wherein the ligand is bound to a positively charged polymeric scaffold. The target cells can be permeabilized using, for example, lysolecithin, TWEEN and its derivatives, NP-40, TRITON X-100, phosphatidylcholine or phospholipases, lysophospholipids containing 1-acyl-sn-glycero-3-phosphate esters of ethanolamine, choline, serine or threonine, the Lauroyl and myristoyl derivatives of lysophosphatidylcholine, lysophosphatidylserine or lysophosphatidylthreonine or imidazoline derivatives or dodecyl phosphocholine, NP-40 and its derivatives, SDS and SLS and their derivatives. Target cells also can be permeabilized by osmotic shock or high-voltage electric pulses.

Methods of the invention can be performed in vivo or ex vivo. For ex vivo transfection, the target cells are removed from a subject, for example, by a biopsy procedure. An appropriate ligand is selected based on knowledge of a cell surface receptor, for example, expressed by the target cell. The target cells can be permeabilized, if desired, and contacted with a composition comprising an exogenous nucleic acid molecule, a liposome, a ligand and a polymeric scaffold, wherein the ligand can bind to a cell surface receptor. The transfected cells can then be implanted into a subject, generally the subject from whom the cells originally were obtained. Thus, the invention provides a means of performing ex vivo gene therapy.

A method of the invention is exemplified by the introduction of an exogenous nucleic acid molecule encoding a protein, for example, beta-galactosidase or Kras-2b, into primary perichondrial cells in vivo or ex vivo. In one aspect, high transfection efficiency of about 70% or greater is obtained. The invention also provides methods of introducing an exogenous nucleic acid molecule into a target cell in vivo by directly injecting a composition of the invention, containing the exogenous nucleic acid molecule, into the desired site in a subject. For example, a composition of the invention can be administered directly into the articular region of a knee joint having an osteochondral defect and, if desired, a cell permeabilizing agent also can be administered. Cells can be permeabilized in vivo by using detergents that are tethered to a ligand-positively charged polymeric scaffold complex. Specific enzymes that result in permeabilizing the cell, such as phospholipases can also be used. Toxins that disrupt the membrane can also be used to selectively create pores in the cell membrane. The in vivo methods can be useful for effecting high transfection efficiency of an exogenous nucleic acid molecule into particular cells in a subject. In particular, where the selected target cell expresses a unique cell surface receptor, the ligand can be selected such that only the desired target cells are transfected. Thus, the compositions and methods of the invention provide a means for high transfection efficiency of an exogenous nucleic acid molecule into primary cells, and are thus useful for in vivo and ex vivo gene therapy.

Viral Gene Therapy

References describing a variety of viral delivery vectors are known in the art, some of which are cited herein. Such vectors include, for example, viral vectors (such as adeno-associated viruses (AAV), liposomes and other lipid-containing complexes, and other macromolecular complexes capable of mediating delivery of a polynucleotide to a host cell. As described above and in the cited references, vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., Lupton, S., WO 92/08796, published 29 May 1992; and Lupton, S., WO 94/28143, published 8 Dec. 1994). Such marker genes can provide an added measure of control that can be advantageous in gene therapy contexts. A large variety of such vectors is known in the art and are generally available (see, e.g., the various references cited above).

Additional references describing adenovirus vectors and other viral vectors which could be used in the methods of the present invention include the following: Horwitz, M. S., Adenoviridae and Their Replication, in Fields, B., et al. (eds.) Virology, Vol. 2, Raven Press New York, pp. 1679-1721, 1990); Graham, F., et al., pp. 109-128 in Methods in Molecular Biology, Vol. 7: Gene Transfer and Expression Protocols, Murray, E. (ed.), Humana Press, Clifton, N.J. (1991); Miller, N., et al., FASEB Journal 9: 190-199, 1995; Schreier, H, Pharmaceutica Acta Helvetiae 68: 145-159, 1994; Schneider and French, Circulation 88:1937-1942, 1993; Curiel D. T., et al., Human Gene Therapy 3: 147-154, 1992; Graham, F. L., et al., WO 95/00655 (5 Jan. 1995); Falck-Pedersen, E. S., WO 95/16772 (22 Jun. 1995); Denefle, P. et al., WO 95/23867 (8 Sep. 1995); Haddada, H. et al., WO 94/26914 (24 Nov. 1994); Perricaudet, M. et al., WO 95/02697 (26 Jan. 1995); Zhang, W., et al., WO 95/25071 (12 Oct. 1995). A variety of adenovirus plasmids are also available from commercial sources, including, e.g., Microbix Biosystems of Toronto, Ontario (see, e.g., Microbix Product Information Sheet: Plasmids for Adenovirus Vector Construction, 1996).

Additional references describing AAV vectors which could be used in the methods of the present invention include the following: Carter, B., Handbook of Parvoviruses, vol. I, pp. 169-228, 1990; Berns, Virology, pp. 1743-1764 (Raven Press 1990); Carter, B., Curr. Opin. Biotechnol., 3: 533-539, 1992; Muzyczka, N., Current Topics in Microbiology and Immunology, 158: 92-129, 1992; Flotte, T. R., et al., Am. J. Respir. Cell Mol. Biol. 7:349-356, 1992; Chatterjee et al., Ann. NY Acad. Sci., 770: 79-90, 1995; Flotte, T. R., et al., WO 95/13365 (18 May 1995); Trempe, J. P., et al., WO 95/13392 (18 May 1995); Kotin, R., Human Gene Therapy, 5: 793-801, 1994; Flotte, T. R., et al., Gene Therapy 2:357-362, 1995; Allen, J. M., WO 96/17947 (13 Jun. 1996); and Du et al., Gene Therapy 3: 254-261, 1996.

Additional references describing non-viral vectors which could be used in the methods of the present invention include the following: Ledley, F D, Human Gene Therapy 6: 1129-1144, 1995; Miller, N., et al., FASEB Journal 9: 190-199, 1995; Chonn, A., et al., Curr. Opin. in Biotech. 6: 698-708, 1995; Schofield, J P, et al., British Med. Bull. 51: 56-71, 1995; Brigham, K. L., et al., J. Liposome Res. 3: 31-49, 1993; Brigham, K. L., WO 91/06309 (16 May 1991); Felgner, P. L., et al., WO 91/17424 (14 Nov. 1991); Solodin et al., Biochemistry 34: 13537-13544, 1995; WO 93/19768 (14 Oct. 1993); Debs et al., WO 93/25673; Felgner, P. L., et al., U.S. Pat. No. 5,264,618 (Nov. 23, 1993); Epand, R. M., et al., U.S. Pat. No. 5,283,185 (Feb. 1, 1994); Gebeyehu et al., U.S. Pat. No. 5,334,761 (Aug. 2, 1994); Feigner, P. L., et al., U.S. Pat. No. 5,459,127 (Oct. 17, 1995); Overell, R. W., et al., WO 95/28494 (26 Oct. 1995); Jessee, WO 95/02698 (26 Jan. 1995); Haces and Ciccarone, WO 95/17373 (29 Jun. 1995); Lin et al., WO 96/01840 (25 Jan. 1996).

For purposes of illustrating vector-mediated gene delivery of Kras-2b to the tissues of the joint, an adenovirus vector can be constructed by the rescue recombination technique as described in McGrory W J, et al., Virology 163:614-617, 1988. Briefly, the transgene of interest is cloned into a shuttle vector that contains a promoter, polylinker and partial flanking adenovirus sequences from which E1A/E1B genes have been deleted.

Illustrative shuttle vectors include, e.g., plasmid “pAC1” (Virology 163:614-617, 1988) (or an analog) which encodes portions of the left end of the human adenovirus 5 genome but lacks the early protein region comprising E1A and E1B sequences that are essential for viral replication; and plasmid “ACCMVPLPA” (J Biol Chem 267:25129-25134, 1992) which contains a polylinker, CMV promoter and SV40 polyadenylation signal flanked by partial adenovirus sequences from which the E1A/E1B genes have been deleted. The use of plasmids such as pAC1 or ACCMVPLA can thus facilitate the cloning process.

The shuttle vector can then be co-transfected, along with a plasmid comprising the entire human adenovirus 5 genome (but with a length too large to be encapsidated), into suitable host cells such as human 293 cells. Co-transfection can be conducted by calcium phosphate precipitation or lipofection (see, e.g., Biotechniques 15:868-872, 1993).

As an illustrative plasmid for co-transfection, plasmid “JM17” encodes the entire human adenovirus 5 genome plus portions of the vector pBR322 including the gene for ampicillin resistance (4.3 kb) (Giordano, et al. Nature Medicine 2: 534-539, 1996). Although JM17 encodes all of the adenovirus proteins necessary to make mature viral particles, it is too large to be encapsidated (40 kb versus 36 kb for wild type).

In a small subset of co-transfected cells, “rescue recombination” occurs between the transgene-containing shuttle vector (such as plasmid pAC1) and the plasmid having the entire adenovirus 5 genome (such as plasmid pJM17) which generates a recombinant genome that contains the transgene of interest in place of the deleted E1A/E1B sequences, and that secondarily loses the additional sequence (such as pBR322 sequences) during recombination, thereby being small enough to be encapsidated (see, e.g., Giordano, et al. Nature Medicine 2: 534-539, 1996). The CMV driven β-galactosidase gene in adenovirus HCMVSP1lacZ (Nature Medicine 2: 534-539, 1996) can be used to evaluate the efficiency of gene transfer using X-gal treatment.

A variety of other vectors suitable for in vivo gene therapy can also be readily employed to deliver Kras-2b transgenes in accordance with the present invention. Such other vectors include, by way of illustration, other viral vectors such as adeno-associated virus (AAV) vectors; non-viral protein-based delivery platforms); as well as lipid-based vectors (including, e.g., cationic liposomes and analogous gene delivery complexes. The preparation and use of these and other vectors are described in the art (see, e.g., the references regarding gene delivery vectors cited above).

The present invention contemplates the use of cell targeting not only by delivery of the transgene into the joint of a mammal, for example, but also by use of targeted vector constructs having features that tend to target gene delivery and/or gene expression to particular host cells or host cell types (such as the perichondrium). Such targeted vector constructs would thus include targeted delivery vectors and/or targeted vectors, as described in more detail below and in the published art. Restricting delivery and/or expression can be beneficial as a means of further focusing the potential effects of gene therapy. The potential usefulness of further restricting delivery/expression depends in large part on the type of vector being used and the method and place of introduction of such vector. As described herein, delivery of vectors via intra-synovial injection to the joint has been observed to provide, in itself, highly targeted gene delivery (see the Examples below). In addition, using vectors that do not result in transgene integration into a replicon of the host cell (such as adenovirus and numerous other vectors), perichondrial cells are expected to exhibit relatively long transgene expression since certain cells such as chrondrocytes do not undergo rapid turnover. In contrast, expression in more rapidly dividing cells would tend to be decreased by cell division and turnover. However, other means of limiting delivery and/or expression can also be employed, in addition to or in place of the illustrated delivery method, as described herein.

Targeted delivery vectors include, for example, vectors (such as viruses, non-viral protein-based vectors and lipid-based vectors) having surface components (such as a member of a ligand-receptor pair, the other half of which is found on a host cell to be targeted) or other features that mediate preferential binding and/or gene delivery to particular host cells or host cell types. As is known in the art, a number of vectors of both viral and non-viral origin have inherent properties facilitating such preferential binding and/or have been modified to effect preferential targeting (see, e.g., Miller, N., et al., FASEB Journal 9: 190-199, 1995; Chonn, A., et al., Curr. Opin. in Biotech. 6: 698-708, 1995; Schofield, J P, et al., British Med. Bull. 51: 56-71, 1995; Schreier, H, Pharmaceutica Acta Helvetiae 68: 145-159, 1994; Ledley, FD, Human Gene Therapy 6: 1129-1144, 1995; Conary, J. T., et al., WO 95/34647 (21 Dec. 1995); Overell, R. W., et al., WO 95/28494 (26 Oct. 1995); and Truong, V. L. et al., WO 96/00295 (4 Jan. 1996)).

Targeted vectors include vectors (such as viruses, non-viral protein-based vectors and lipid-based vectors) in which delivery results in transgene expression that is relatively limited to particular host cells or host cell types. By way of illustration, Kras-2b transgenes to be delivered according to the present invention can be operably linked to heterologous tissue-specific promoters thereby restricting expression to cells in that particular tissue.

Recombinant viral vectors, such as adenoviral vectors, can be plaque purified according to standard methods. By way of illustration, recombinant adenoviral viral vectors can be propagated in human 293 cells (which provide E1A and E1B functions in trans) to titers in the preferred range of about 1010-1012 viral particles/ml.

Propagation and purification techniques have been described for a variety of viral vectors that can be used in conjunction with the present invention. Adenoviral vectors are exemplified herein but other viral vectors such as AAV can also be employed. For adenovirus, cells can be infected at about 80% confluence and harvested 48 hours later. After 3 freeze-thaw cycles the cellular debris can be collected by centrifugation and the virus purified by CsCl gradient ultracentrifugation (double CsCl gradient ultracentrifugation is preferred).

Prior to in vivo injection, the viral stocks can be desalted by gel filtration through Sepharose columns such as G25 Sephadex. The product can then be filtered through a 30 micron filter, thereby reducing the potential for deleterious effects associated with injection of unfiltered virus. The resulting viral stock preferably has a final viral titer that is at least about 10¹⁰-10¹² viral particles/ml.

Preferably, the recombinant adenovirus is highly purified, and is substantially free of wild-type (potentially replicative) virus. For these reasons, propagation and purification can be conducted to exclude contaminants and wild-type virus by, for example, identifying successful recombinants with PCR using appropriate primers, conducting two rounds of plaque purification, and double CsCl gradient ultracentrifugation.

The means and compositions which are used to deliver the vectors carrying Kras-2b transgenes depend on the particular vector employed as is well known in the art. Typically, however, a vector can be in the form of an injectable preparation containing pharmaceutically acceptable carrier/diluent such as saline, for example.

For viral vectors (such as adenovirus), the final titer of the virus in the injectable preparation is preferably in the range of about 10⁷-10¹³ viral particles which allows for effective gene transfer. Other pharmaceutical carriers, formulations and dosages are described below.

Vectors comprising Kras-2b transgenes can be delivered to the joint by direct intra-synovial injection, in an amount sufficient for the transgene to be expressed and to provide a therapeutic benefit. Such an injection is preferably made deeply into the synovial space to provide general distribution to all areas of the joint.

Transgenic Non-Human Animals

The invention provides transgenic non-human animals comprising a nucleic acid construct of the invention, including an expression cassette or vector or a transfected or transformed cell comprising a nucleic acid expressing Kras-2b operably linked to promoter and/or enhancer. The invention also provides methods of making and using these transgenic non-human animals.

The transgenic non-human animals can be, e.g., mice, rats, rabbits, dogs, cats, cows, goats, sheep, pigs, horses, and monkeys, comprising a nucleic acid construct of the invention. These animals can be used, e.g., as in vivo models for Kras-2b expression and activity, e.g., as models to screen for compound that can activate Kras-2b gene activity in vivo.

The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP).

“Knockout animals” can also be used to practice the methods of the invention. For example, in one aspect, the transgenic or modified animals of the invention comprise a e.g., the endogenous Kras-2b gene locus, or subsequences thereof. “Knockouts” can be prepared by deletion or disruption by homologous recombination of an endogenous promoter. Homologous recombination and other means to alter (and “knockout”) expression of endogenous sequences is well known in the art and is described in, e.g., U.S. Pat. Nos. 5,464,764; 5,631,153; 5,487,992; 5,627,059, and 5,272,071.

Animal Model of Osteoarthritis

Important prerequisites for developing any osteoarthritis gene therapy technique to be applicable to humans are: (a) constitution of a large animal model that is applicable to clinical joint degeneration and which can provide useful data regarding mechanisms for altered Kras-2b signaling in the setting of osteoarthritis, and (b) accurate evaluation of the effects of gene transfer.

Employed herein is an art-recognized rabbit model of osteoarthritis. In the laboratory, OA has been successfully modeled in rabbits by transecting the anterior cruciate ligament (ACLT). Numerous independent studies have shown that the experimentally induced OA in the rabbit demonstrates the same pathological progression as in humans. The ACLT model is incrementally degradative showing continuing and increasing lesions of cartilage matrix, diminution of cartilage GAGs and decreases in cartilage height starting from 4 to 12 weeks. This cartilage degradation progressively worsens, resulting in complete focal depletion of the cartilage layer by 12 weeks post-ACLT. The effect on subchondral bone density and osteophyte formation also parallels those observed in human OA (Takahashi, et al. (1999) Osteoarthritis and Cartilage, 7:182-190; Setton, et al., (1999) Osteoarthritis and Cartilage, 7:2-14; Goomer, et al., (2005) Clin. Orthop. Rel. Res., 434:239-245).

Our studies with rabbits, described and illustrated in more detail below, demonstrate that over-expression of a Kras-2b by in vivo delivery to the perichondrium, can prevent degradation of cartilage in an animal model predictive of osteoarthritis in humans.

Kits and Libraries

The invention provides kits comprising compositions and methods of the invention, including cells comprising heterologous Kras-2b expression vehicles, transfecting agents, transducing agents, instructions (regarding the methods of the invention), or any combination thereof. As such, kits, cells, vectors and the like are provided herein.

Pharmaceutical Compositions

The invention provides pharmaceutical compositions comprising a Kras-2b-expressing nucleic acid (e.g., a vector, virus, and the like) and a pharmaceutically acceptable excipient. The invention provides parenteral formulations comprising Kras-2b protein or nucleic acids expressing Kras-2b. The invention provides methods for preventing cartilage degradation in a joint or for normalizing cartilage formation in a joint comprising providing a pharmaceutical composition comprising a Kras-2b-expressing nucleic acid; and administering an effective amount of the pharmaceutical composition to a subject in need thereof. In certain aspects of the invention, the subject in need of treatment has or is at risk of developing osteoarthritis.

These methods can be practiced in vivo, ex vivo or in vitro.

The pharmaceutical compositions used in the methods of the invention can be administered by any means known in the art, e.g., parenterally, topically, orally, or by local administration, such as by aerosol or transdermally. In one aspect, the pharmaceutical composition in administered into the synovial space of the joint. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Pharmaceutical formulations can be prepared according to any method known to the art for the manufacture of pharmaceuticals. Such drugs can contain sweetening agents, flavoring agents, coloring agents and preserving agents. A formulation can be admixed with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a chimeric polypeptide or peptidomimetic of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration of hydrophobic active agents of the invention. Oil-based suspensions can be formulated by suspending an active agent (e.g., a chimeric composition of the invention) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical compositions of the invention can be formulated with a permeabilizing agent as described supra, and/or can be administered in conjunction with a permeabilizing agent. By “in conjunction with”, it is meant the nucleic acid is administered before, simultaneously with, or after the permeabilizing agent.

In the methods of the invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In the methods of the invention, the pharmaceutical compounds can be delivered by transdermally, by a topical route, formulated as applicator sticks, solutions, suspensions, emulsions, gels, creams, ointments, pastes, jellies, paints, powders, and aerosols.

In the methods of the invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In the methods of the invention, the pharmaceutical compounds can be parenterally administered, such as by intravenous (IV) administration or administration into a body cavity (e.g., the synovial space) or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides.

Test Molecules and Candidate Molecules

The Kras-2b (synonymous with Kras-4-b) (or its upstream or downstream signaling molecule(s)) nucleic acid or protein is contacted with one or more test molecules to identify candidate molecules that modulate a biological activity of the nucleic acid. Molecules often are organic or inorganic compounds having a molecular weight of 10,000 grams per mole or less, and sometimes having a molecular weight of 5,000 grams per mole or less, 1,000 grams per mole or less, or 500 grams per mole or less. Also included are salts, esters, and other pharmaceutically acceptable forms of the compounds. Compounds that interact with nucleic acids are known in the art (see, e.g., Hurley, Nature Rev. Cancer 2:188-200 (2002); Anantha, et al., Biochemistry Vol. 37, No. 9:2709-2714 (1998); and Ren, et al., Biochemistry 38:16067-16075 (1999)).

Compounds can be obtained using known combinatorial library methods, including spatially addressable parallel solid phase or solution phase libraries; synthetic library methods requiring deconvolution; “one-bead one-compound” library methods; and synthetic library methods using affinity chromatography selection. Examples of methods for synthesizing molecular libraries are described, for example, in DeWitt, et al., Proc. Natl. Acad. Sci. U.S.A. 90:6909 (1993); Erb, et al., Proc. Natl. Acad. Sci. USA 91:11422 (1994); Zuckermann, et al., J. Med. Chem. 37:2678 (1994); Cho, et al., Science 261:1303 (1993); Carrell, et al., Angew. Chem. Int. Ed. Engl. 33:2059 (1994); Carell, et al., Angew. Chem. Int. Ed. Engl. 33:2061 (1994); and Gallop, et al., J. Med. Chem. 37:1233 (1994).

In addition to an organic and inorganic compound, a molecule sometimes is a nucleic acid, a catalytic nucleic acid (e.g., a ribozyme), an inhibitory RNA (RNAi or siRNA), a nucleotide, a nucleotide analog, a polypeptide, an antibody, or a peptide mimetic. Methods for making and using these molecules are known. For example, methods for making ribozymes and assessing ribozyme activity are described (see e.g., U.S. Pat. Nos. 5,093,246; 4,987,071; and 5,116,742; Haselhoff & Gerlach, Nature 334:585-591 (1988) and Bartel & Szostak, Science 261:1411-1418 (1993)). Also, methods for generating siRNA are known (see e.g., Elbashir, et al., Methods 26:199-213 (2002) and http address www.dharmacon.com) and peptide mimetic libraries are described (see, e.g., Zuckermann, et al., J. Med. Chem. 37:2678-2685 (1994)).

In certain embodiments, the activity of a protein or its target nucleic acid sequence is down-regulated, or entirely inhibited, by the use of antisense polynucleotide or inhibitory small nuclear RNA (snRNA), i.e., a nucleic acid complementary to, and which can preferably hybridize specifically to, a coding mRNA nucleic acid sequence, e.g., a mRNA or a subsequence thereof. Binding of the antisense polynucleotide to the mRNA reduces the translation and/or stability of the mRNA.

In the context of this invention, antisense polynucleotides can comprise naturally occurring nucleotides, or synthetic species formed from naturally occurring subunits or their close homologs. Antisense polynucleotides may also have altered sugar moieties or inter-sugar linkages. Exemplary among these are the phosphorothioate and other sulfur containing species which are known for use in the art. Analogs are comprised by this invention so long as they function effectively to hybridize with nucleotides of the invention. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single stranded nucleic acid sequence (either RNA or DNA) capable of binding to target mRNA (sense) or DNA (antisense) sequences for cancer molecules. Antisense or sense oligonucleotides, according to the present invention, comprise a fragment generally at least about 12 nucleotides, preferably from about 12 to 30 nucleotides. The ability to derive an antisense or a sense oligonucleotide, based upon a cDNA sequence encoding a given protein is described in, e.g., Stein &Cohen (Cancer Res. 48:2659 (1988 and van der Krol et al. (Bio Techniques 6:958 (1988)).

In addition to antisense polynucleotides, ribozymes can be used to target and inhibit transcription of protein-encoding nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al., Adv. in Pharmacology 25: 289-317 (1994) for a general review of the properties of different ribozymes).

The general features of hairpin ribozymes are described, e.g., in Hampel et al., Nucl. Acids Res. 18:299-304 (1990); European Patent Publication No. 0360257; U.S. Pat. No. 5,254,678. Methods of preparing are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5: 1151-120 (1994); and Yamada et al., Virology 205:121-126 (1994)).

Biological Activity of Candidate Molecules

Determining whether the biological activity of a kras-2b nucleotide or protein or effector is modulated in a cell, tissue, or organism can be accomplished by monitoring modulation of a signal in an in vitro or in vivo assay. The signal in the assay sometimes is generated or modulated by transcription of the nucleic acid, binding of a protein to the nucleic acid, a fluorophore incorporated in the nucleic acid, or cell proliferation, for example. Transcription can be detected, for example, by directly detecting RNA transcripts or detecting polypeptides translated by transcripts, which are methods known in the art.

Candidate molecules may be screened in in vitro or in vivo assays to determine whether they modulate the biological activity of the nucleic acid. Candidate molecules and nucleic acids can be added to an assay system in any order to determine whether the candidate molecule modulates the biological activity of the nucleic acid. For example, a candidate molecule sometimes is added to an assay system before, simultaneously, or after a nucleic acid is added.

In these assays, candidate molecules are contacted with the nucleic acid in the assay system, where the term “contacting” refers to placing a candidate molecule in close proximity to a nucleic acid and allowing the assay components to collide with one another, often by diffusion. Contacting these assay components with one another can be accomplished by adding them to a body of fluid or in a reaction vessel, for example. The components in the system may be mixed in variety of manners, such as by oscillating a vessel, subjecting a vessel to a vortex generating apparatus, repeated mixing with a pipette or pipettes, or by passing fluid containing one assay component over a surface having another assay component immobilized thereon, for example.

As used herein, the term “system” refers to an environment that receives the assay components, which includes, for example, microtiter plates (e.g., 96-well or 384-well plates), silicon chips having molecules immobilized thereon and optionally oriented in an array (see, e.g., U.S. Pat. No. 6,261,776 and Fodor, Nature 364:555-556 (1993)), and microfluidic devices (see, e.g., U.S. Pat. Nos. 6,440,722; 6,429,025; 6,379,974; and 6,316,781). The system can include attendant equipment for carrying out the assays, such as signal detectors, robotic platforms, and pipette dispensers.

One or more assay components (e.g., the nucleic acid, candidate molecule or nucleic acid binding protein) sometimes are immobilized to a solid support. The attachment between an assay component and the solid support often is covalent and sometimes is non-covalent (see, e.g., U.S. Pat. No. 6,022,688 for non-covalent attachments) and the attachment sometimes is by a linking moiety. The solid support often is one or more surfaces of the system, such as one or more surfaces in each well of a microtiter plate, a surface of a silicon wafer, a surface of a bead (see, e.g., Lam, Nature 354: 82-84 (1991)) optionally linked to another solid support, or a channel in a microfluidic device, for example. Types of solid supports, linker molecules for covalent and non-covalent attachments to solid supports, and methods for immobilizing nucleic acids and other molecules to solid supports are known (see, e.g., U.S. Pat. Nos. 6,261,776; 5,900,481; 6,133,436; and 6,022,688; and WIPO publication WO 01/18234).

Protein molecules sometime are contacted with the nucleic acid. Polypeptide molecules sometimes are added to the system in free form, and sometimes are linked to a solid support or another molecule. For example, polypeptide test molecules sometimes are linked to a phage via a phage coat protein. The latter embodiment often is accomplished by using a phage display system, where nucleic acids linked to a solid support are contacted with phages that display different polypeptide candidate molecules. Phages displaying polypeptide candidate molecules that interact with the immobilized nucleic acids adhere to the solid support, and phage nucleic acids corresponding to the adhered phages then are isolated and sequenced to determine the sequence of the polypeptide test molecules that interacted with the immobilized nucleic acids. Methods for displaying a wide variety of peptides or proteins as fusions with bacteriophage coat proteins are known (Scott and Smith, Science 249:386-390 (1990); Devlin, Science 249:404-406 (1990); Cwirla, et al., Proc. Natl. Acad. Sci. 87:6378-6382 (1990); Felici, J. Mol. Biol. 222:301-310 (1991); U.S. Pat. Nos. 5,096,815 and 5,198,346; U.S. Pat. Nos. 5,223,409; 5,403,484; 5,571,698; and 5,766,905). Methods also are available for linking the test polypeptide to the N-terminus or the C-terminus of the phage coat protein.

A signal generated by the system when a candidate molecule binds to a nucleic acid and/or a nucleic acid binding protein often scales directly with a range of increasing nucleic acid, nucleic acid binding protein, or candidate molecule concentrations. Signal intensity often exhibits a hyperbolic relationship when plotted as a function of nucleic acid, candidate molecule, or nucleic acid binding protein concentrations. The signal sometimes is increased relative to background signal levels when a candidate molecule binds to a nucleic acid and/or a nucleic acid binding protein, and sometimes the signal decreases relative to background signal levels under such circumstances. The candidate molecules often interact with the nucleic acid and/or nucleic acid binding protein by reversible binding, and sometimes interact with irreversible binding. For example, the candidate molecule may reversibly form a covalent bond between a portion of the candidate molecule and an amino acid side chain in the protein (e.g., a lysine), depending on the chemical structure of the candidate molecule.

Candidate molecules often are identified as interacting with the nucleic acid and/or nucleic acid binding protein when the signal produced in a system containing the candidate molecule is different than the signal produced in a system not containing the candidate molecule. While background signals may be assessed each time a new candidate molecule, nucleic acid, or nucleic acid binding protein is probed by the assay, detecting the background signal often is not required each time a new test molecule or test nucleic acid is assayed. Control assays also can be performed to determine background signals and to rule out false positive results and false negative results. Such control assays often do not comprise one or more assay components included in other assays (e.g., a control assay sample sometimes does not include a candidate molecule, a nucleic acid, or a protein that interacts with the nucleic acid).

In addition to determining whether a candidate molecule gives rise to a different signal, the affinity of the interaction between the candidate molecule with the nucleic acid and/or nucleic acid binding protein sometimes is quantified. IC₅₀, K_{d}, or Kᵢ threshold values sometimes are compared to the measured IC₅₀ or K_{d} values for each interaction, and thereby are used to identify a candidate molecule that interacts with the nucleic acid or nucleic acid binding protein and modulates the biological activity. For example, IC₅₀ or K_{d} threshold values of 10 M or less, 1 M or less, and 100 nM or less often are utilized, and sometimes threshold values of 10 nM or less, 1 nM or less, 100 pM or less, and 10 pM or less are utilized to identify candidate molecules that interact with nucleic acids and/or binding proteins and modulate the biological activity.

Specific assays sometimes are utilized to identify candidate molecules that modulate the biological activity of a nucleic acid and/or binding protein complex. For example, fluorescence assays, gel mobility shift assays (see, e.g., Jin & Pike, Mol. Endocrinol. 10:196-205 (1996) and Postel, J. Biol. Chem. 274:22821-22829 (1999)), polymerase arrest assays, transcription reporter assays, DNA cleavage assays, protein binding and apoptosis assays (see, e.g., Amersham Biosciences (Piscataway, N.J.)) sometimes are utilized. Also, topoisomerase assays sometimes are utilized subsequently to determine whether the quadruplex interacting molecules have a topoisomerase pathway activity (see, e.g., TopoGEN, Inc. (Columbus, Ohio)).

Use of Kras Effectors

In addition, some of the kras effectors in the signal transduction cascade may also form good therapeutic candidates. A point mutant of Kras 2b was identified as a potent oncogene. While 30% of all carcinomas carry a ras mutation, 90% of pancreatic adenocarcinomas are associated with an oncogenic mutation in the kras gene. Kras mutations are also strongly associated with lung carcinogenesis. N-Ras, H-Ras, and Kras-2A isoforms also differ from KRas-2B, with respect to their respective downstream modifications and subcellular localization via scaffolds. For example, mammalian MAPK scaffolds appear to have specific subcellular localizations and are now recognized to be integral to compartmentalized ras mediated signaling. This compartmentalization also explains the diversity of ras-specific responses observed in vivo. Other examples are the Erk scaffolds such as the kinase suppressor of Ras (KSR), which is a positive regulator of the Ras/MAPK pathway. KSR is a multidomain protein that binds Raf-1, MEK, and Erk, as well as several other proteins. In resting cells, it is sequestered in the cytosol, like by 14-3-3 proteins. However, in response to mitogenic signals, KSR becomes dephosphorylated at S392, loses affinity for 14-3-3, and translocates to the plasma membrane by virtue of a cysteine-rich domain. Thus, KSR represents an inducible scaffold for Ras/MAPK signaling with specificity for the plasma membrane. There are at least two ksr genes in mammals. Targeted disruption of ksr1 yielded mice that were relatively normal, although T-cell activation was impaired. MEK partner 1 (MP1) binds MEK1 and Erk1 but not MEK2 and Erk2 and that binding facilitated the phosphorylation of Erk1 by MEK1. (Mor & Philips (2006) Annu. Rev. Immunol. 24:771-800.)

Caveolin inhibited H-Ras but not KRas signaling and this differential effect could be mimicked by cholesterol depletion. These data pointed to the fact that ras isoforms operate in functionally distinct plasma membrane microdomains. Additional data showed that while H-Ras was associated with lipid rafts, K-Ras was not. Moreover, access of H-Ras to lipid rafts was dynamic; GDP-bound H-Ras was favored, suggesting that activation takes place in rafts, but effector engagement occurs in nonraft domains. These studies confirmed that whereas GDP bound H-Ras clustered in domains that were sensitive to cholesterol depletion, GTP-bound H-Ras and all forms of K-Ras clustered in domains that were insensitive to cholesterol depletion.

As such, other ras molecules. including but not limited to H, N, K, and dominant negative, site-directed mutants of ras or Kras, truncations of ras and RAF, RAP, RAC, PAC, 14-3-3, MAPK, MAPKK (or variants), MAPKKK (or variants), MEK½, ERK 1/2, mTOR, TOR, Tob1, P38, PI3K, Ink4, Akt, JNK, S6, S6 kinases, cyclins, Smads and other players in the ras signal transduction cascade are useful in the current invention for treating and/or preventing joint diseases and disorders related to arthritis and inflammation. In one aspect, these molecules (and modulatory compounds thereof) are useful for treating or preventing osteoarthritis. In other aspects, these molecules (and modulatory compounds thereof) are useful for treating or preventing Rheumatoid arthritis (RA), Crystal disease, Coeliac disease, diabetes mellitus type 1 (IDDM), systemic lupus erythematosus (SLE), Sjögren's syndrome, multiple sclerosis (MS), Hashimoto's thyroiditis, Graves' disease and idiopathic thrombocytopenic purpura. In certain aspects, the molecules and modulatory compounds thereof treat or prevent the joint disease or disorder by decreasing the cellular proliferation index of one or more tissues of the joint.

The following Examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### EXAMPLES

### Example 1

### General Methods

Gene Delivery Protocol:

A two step gene delivery protocol was used as described elsewhere (Goomer, U.S. Pat. No. 6,573,101; Goomer et al., (2000) Clinical Orthopaedics & Related Research, 379:S189-S200).

DNA components used in these experiments were: 1) a mammalian expression plasmid carrying the bacterial β-galactosidase reporter gene purchased from Promega (the pSV-β-Galactosidase Control Vector; Madison, Wis.), and, 2) the human K-ras2b gene carried on the pORF9-hKRAS2b v21 mammalian expression plasmid (Invivogen, San Diego, Calif.). The expression plasmids were amplified in large quantities from glycerol stocks of transformed DH5α cells using an endotoxin free plasmid DNA isolation and purification system (the endotoxin free Mega-Giga plasmid purification kit, Promega, Inc., Madison, Wis.).

The two-step gene delivery system consists of the following: tissues were treated with lysolecithin as a permeabilizing agent, followed shortly (2-5 minutes later) with the plasmid DNAs condensed with a positively charged polymer. In these examples, poly-L-lysine was covalently bonded to a high-affinity ligand, transferrin, forming the DTPL nano-particles. The DTPL nano-particles were further complexed with cationic liposomes forming the DTPLL nano-particles (See Goomer, U.S. Pat. No. 6,573,101; Goomer et al., (2000) Clinical Orthopaedics & Related Research, 379:S189-S200). Of course, it is well understood that the poly-1-lysine scaffold could be replaced with another suitable positively charged polymer covalently bonded to a high-affinity ligand, while the ligand, transferrin, can be replaced with a different suitable tissue or cell specific ligand(s).

The contra-lateral rabbit knees were used as normal controls.

The amounts of DNAs, the DNA-complexing reagents and permeabilizing agent delivered in the injections are listed in Table II (below).

ACL Transection and Gene Delivery:

All humane-treatment of laboratory animal procedures were followed as approved and directed by the Institutional Review Committee at UCSD. Three mature New Zealand White (NZW) rabbits (n=3) (Age: 10-12 months) with closed epiphysis per group (Table I) were used. Experimental OA was induced by ACL transection in the left knee. ACL transections were performed as follows: animals were sedated and anesthetized; under sterile conditions, a 3-cm medial para-patellar incision was made; the patella was dislocated and the ACL was transected using a size 11 surgical blade. ACL transection was confirmed both visually and by performing Lachman testing.

At this time, 150 micro-liters of the Permeabilizing Agent (PA) (Lysolecithin or L-α-Lysophosphatidylcholine (PC) Sigma-Adrich, St. Louis, Mo.) was added into the intra-articular site. The final injectate concentration of lysolecithin was (0.175% w/v). Exactly 2-minutes following this first injection, a second injection containing 150 micro-liters of the DNA+TPLL (transferring-Poly-L-Lysine and liposome) cocktail were added. The exact concentration of the various reagents is delineated in Table II (Goomer et al., (2000) Clinical Orthopaedics & Related Research, 379:S189-S200). The contra-lateral rabbit knees were used as normal controls. The surgical site was sutured and the animals awakened. Rabbits were given appropriate postoperative care and analgesia for pain. Following recovery, the animals were allowed free mobility and range.

Two weeks following surgery, the rabbit knees (left knee) received an intra-articular injection of the gene delivery cocktail plus DNA, also as detailed in Table II. The 150 micro-liters of the PA were followed 2 minutes later with 150 micro-liters of the DNA cocktail. The two reagents were loaded into the same injection syringe and injected into the joint cavity in reverse order.

Four weeks post-ACLT the experimental animals were euthanized. Any unusual swelling of the knee joint was carefully observed and documented. The articular tissues (synovium, meniscus, femoral condyle, tibial plateau, ACL and the PCL) were immediately harvested from each knee and used for assessments.

TABLE II

| Rabbit | | | | |
|---|---|---|---|---|
| groups* | TPLL | Liposomes** | PA (150μl)*** | DNAs |
| Mock | 0.5 μg | 4:1(Lipo:DNA) | 0.175% (w/v) | pUC19 plasmid |
| group | | by volume (μl) | | 50 μg |
| β-gal. | 0.5 μg | 4:1(Lipo:DNA) | 0.175% (w/v) | β-gal. plasmid |
| group | | by volume (μl) | | 50 μg |
| Kras-2b | 1.0 μg | 4:1(Lipo:DNA) | 0.175% (w/v) | 50 μg Kras 2b |
| group | | by volume (μl) | | plasmid + |
| | | | | 50 μg β-gal. plasmid |

**Liposomes volume: 4 parts liposomes (volume (μl)) to 1 part DNA (volume (μl))
*** Permeabilizing Agent (Lysolecithin (Sigma, St Louis, MO), was injected exactly 2 minutes prior to the injection of the DNA cocktail = DNA + (Tranferrin-poly-l-lysine and liposome complexes) using the same syringe; the syringe was loaded with DNA cocktail first followed with PA and was unloaded in reverse order.

Histological Assessment:

After harvest, the knee tissues (synovium, menisci, femoral condyle, tibial plateau, the ACL and the PCL) were either immersed in a buffered solution of 10% formalin for tissue-fixation for 12-24 hours or used for RT-PCR assessment or treated to assess for the presence of β-galactosidase enzyme activity as detailed below; the histology specimens were then moved into 5% hydrochloric acid for 17 hours to decalcify. The decalcified tissue was then sequentially dehydrated in alcohol and embedded into paraffin blocks and sectioned into 5-micron sections. The sections were stained with Eosin (for synovium and ligaments) and H&E and Safranin-O (for cartilage sections) as per previously published protocols (Goomer et al., (2000) Clinical Orthopaedics & Related Research, 379:S189-S200).

β-Galactosidase Functional Assay:

The level of functional β-galactosidase was determined by enzymatic assays in tissues before paraffin embedding and sectioning as per published protocols (Goomer et al., (2000) Clinical Orthopaedics & Related Research, 379:S189-S200). The periarticular ligaments, synovium and surrounding tissues were stained with X-gal for 12-18 hours at neutral pH. The reaction of bacterially-expressed β-galactosidase enzyme with X-gal produces a blue colored product. The stained tissues were paraffin sectioned and stained with Eosin.

Real Time RT-PCR:

The relative concentrations of Kras-2b, β-galactosidase, S6 and Cyclin D1 transcripts were determined by performing real-time PCR on first strand synthesis products from synovial mRNAs. The tissue RNAs were isolated and purified using standard molecular biology techniques. The first strand synthesis products were synthesized as per previously published protocols (Takahashi, et al. (1999), Osteoarthritis and Cartilage, 7:182-190). Real-time PCR was performed in triplicate in an Applied Biosystems 7300 Real-Time PCR as per manufacturer's protocols using synthetic oligonucleotides specific for the genes in question that were designed using the Primer Express™ software provided by ABI. The oligonucleotides were synthesized by Operon Biosciences. Briefly, the PCR step was performed in a 96 well plate containing ‘PCR master mix’ (containing SYBR green, a buffer, the Taq enzyme, from ABI), primers and an appropriate amount of the first-strand cDNA product. The cDNAs were serially diluted and standardized to U6 snRNA used as an internal standard (Goomer & Kunkel, (1992) Nucleic Acids Res, 20:4903-4912). After running the protocol, data were analyzed using the ABI relative gene expression software.

### Example 2

### Testing the Efficiency of Gene Delivery and Expression in Articular Tissues

A major road-block to the development of effective gene therapy regimes for OA is the apparently low efficiency of gene delivery, in intra-articular tissues in vivo—using the currently available non-viral protocols. It is of utmost importance to develop an efficient non-viral method to deliver therapeutic genes into the articular tissues, in particular, the synovium and the ligaments to affect the OA pathology. Therefore, the efficiency of marker gene delivery into the articular tissues in live animals was studied.

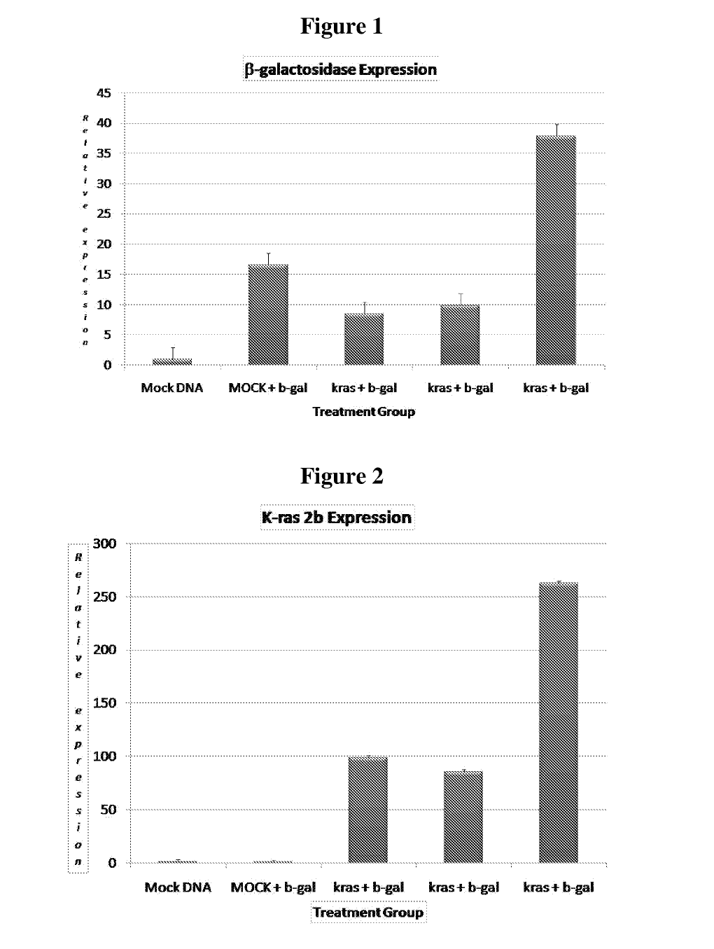

Expression of β-galactosidase marker gene in the synovium was quantified by real-time PCR. Real-time PCR was performed on first-strand synthesis cDNA products synthesized from the RNAs isolated and purified from the synovium tissues isolated from the (β-galactosidase group) and expanded in tissue-culture. The expression of the β-galactosidase gene was significantly increased (ranging from 8× to 39× over base-line) in the synovium of animals transfected with β-galactosidase (FIG. 1). This demonstrated that beta-galactosidase transcripts were being produced robustly in the transfected tissue 4-weeks following gene delivery.

Similarly, Kras-2b (FIG. 2) transcripts were significantly increased in the tissues transfected with the Kras-2b plasmids (ranging from 100× to >250× over base-line).

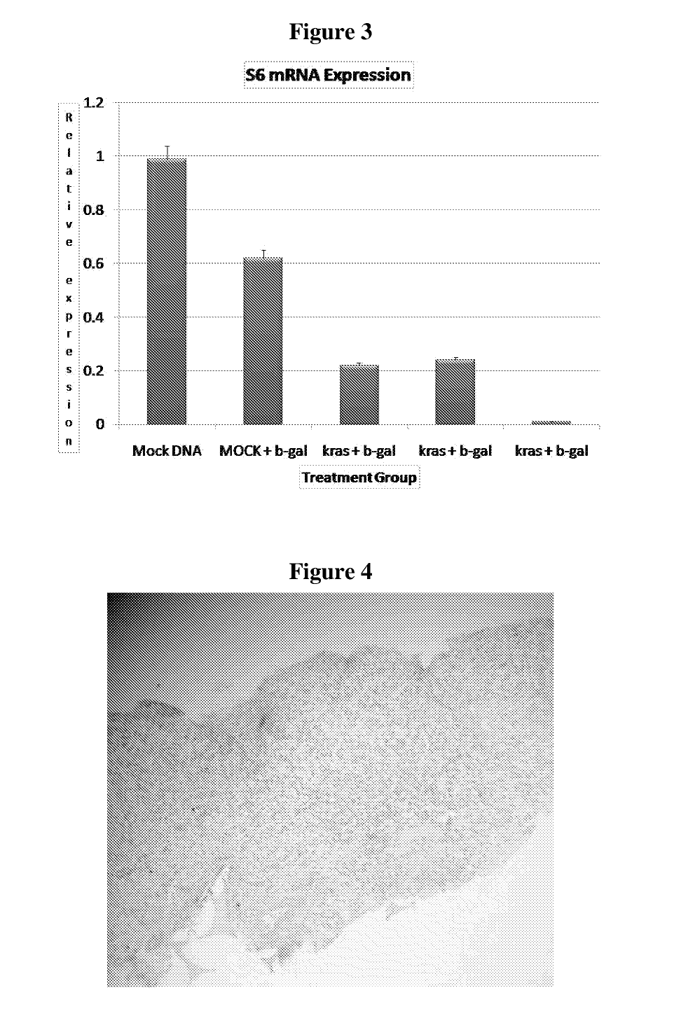

S6 is a ras dependent ribosomal gene whose expression is a bona fide index of cellular proliferation. S6 is required for the passage of the cells through the cell-cycle. Our results clearly show that S6 gene expression is severely repressed in tissues and cells where wild-type Kras is increased. These results further demonstrate a specific and dose-dependent repression of S6 RNAs by Kras-2b in transfected tissues (from 0.2× to 0.01× compared to base-line) (FIG. 3; compare with FIG. 2). Thereby, the proliferation of synovial cells transfected with wild type Kras-2b is severely repressed. Our quantitative data also show that Kras-2b was delivered at functionally high levels in the transduced tissues, in particular, in the synovium for the length of the study period of 28 days (FIGS. 2 and 3). Kras was also found to linearly repress cyclin D₁ gene expression in the synovium (data not shown).

The relative amounts of transfected cells in the tissues of interest were quantified. Tissues isolated from the rabbit knees at 4 weeks post-ACLT were stained with X-gal. The synovial tissues were sectioned and co-stained with eosin (FIG. 4). As observed previously in the canine tendon repair model, the synovial tissues in the rabbit were also labeled at efficiencies approaching 100% (Goomer, et al. (2000) Clinical Orthopaedics & Related Research, 379:S189-S200).

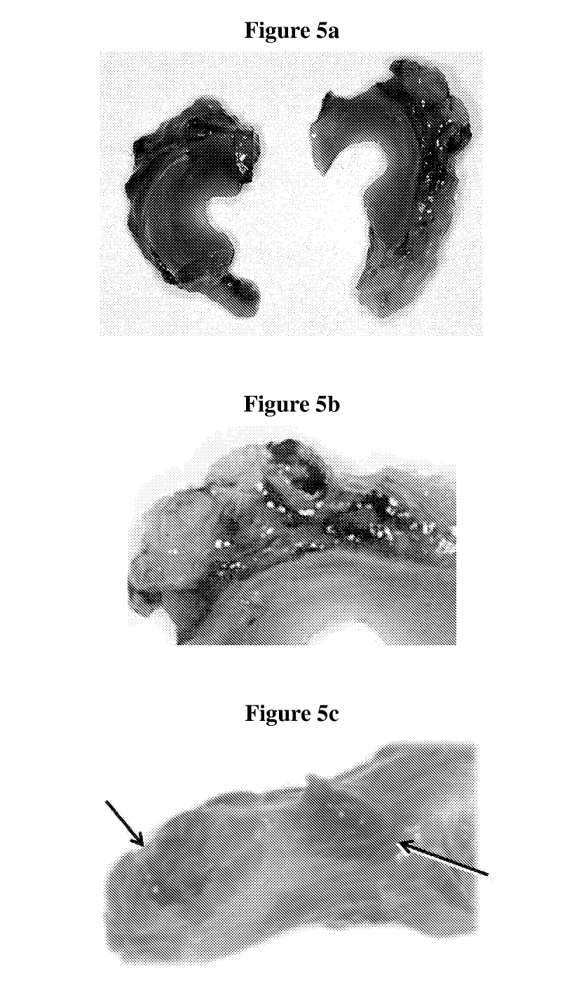

Additionally, the synovial tissues (and fat pad cells) attached to the menisci (FIGS. 5a and 5b) and cells of ACL (FIG. 5c) and PCL (not shown) at the sites of attachment to the bones were also transfected and labeled highly efficiently (FIG. 5c). Thus the results show, for the first time, that knee tissues can be very efficiently transfected by using the proprietary nano-formulated non-viral complex containing plasmids expressing a marker, the bacterial β-galactosidase gene (FIGS. 1-5).

### Example 3

### Kras-2b Prevents the Development of OA Related Pathological Damage

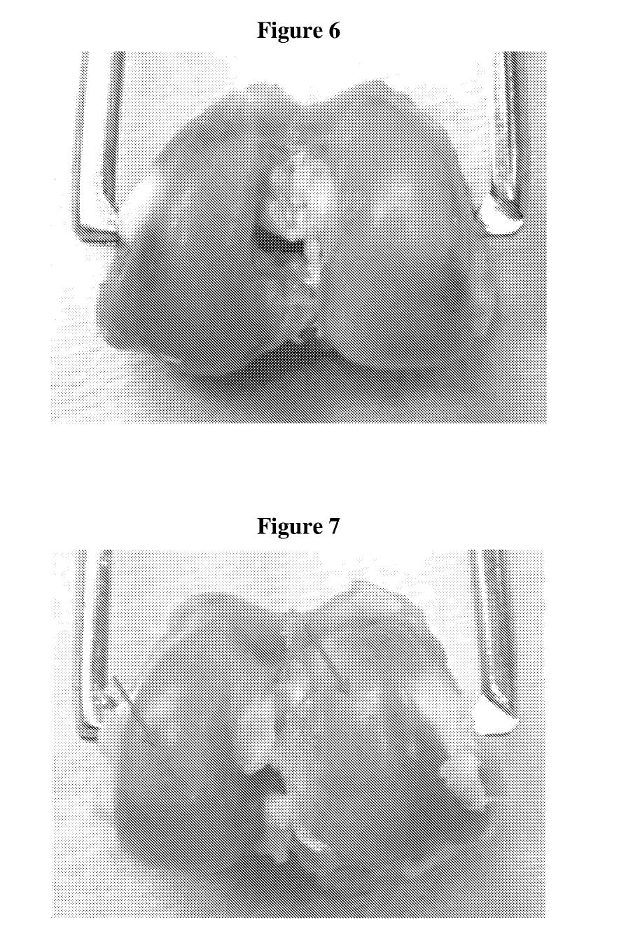

Osteoarthritis was induced in rabbits by completely cutting the ACL (anterior cruciate ligament)(compare FIG. 6 vs. FIG. 7). Rabbit ACLT is a standard and accepted model of knee osteoarthritis and has been used to test other OA drugs (such as SYNVISC and SUPARTZ®) (Goomer, et al., (2005) Clin. Orthop. Rel. Res., 434:239-245). As discussed supra, after surgery, knees were transfected with Kras-2b encoding vectors in a non-viral complex or were transfected with a beta-galactosidase construct.

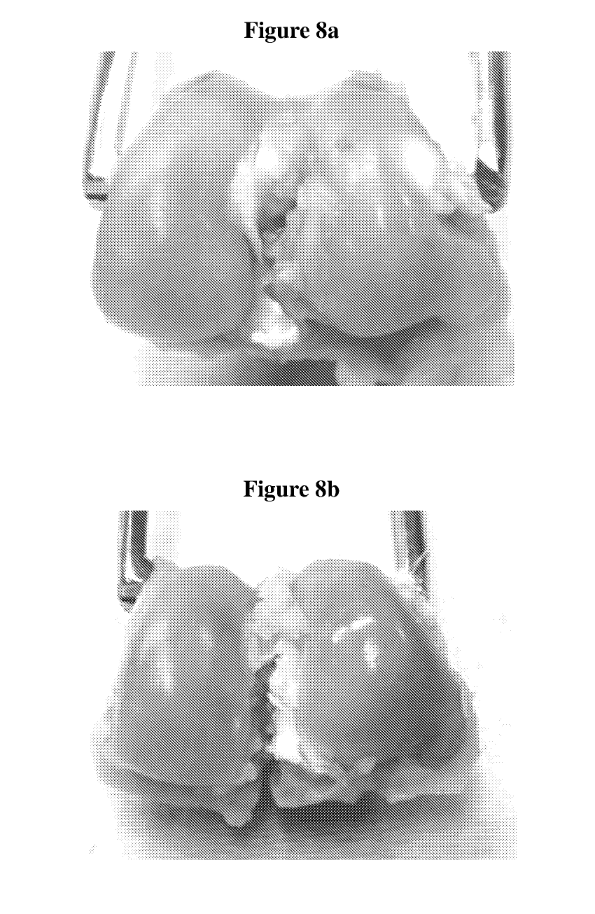

Macroscopic evaluation of cartilaginous tissues of the knees (femoral condyles) transfected with Kras-2b clearly demonstrated that the Kras-2b transfected knees did not develop osteoarthritic lesions at 4-weeks post-ACLT treatment (FIGS. 8 a and b). This was easily observable when comparing the gross morphology of Kras-2b femoral condyles with the 4 weeks post-ACLT animal knees receiving the marker gene (FIG. 7). In the control knees, cartilage fibrillations and surface roughness are indicated with arrows (FIG. 7).

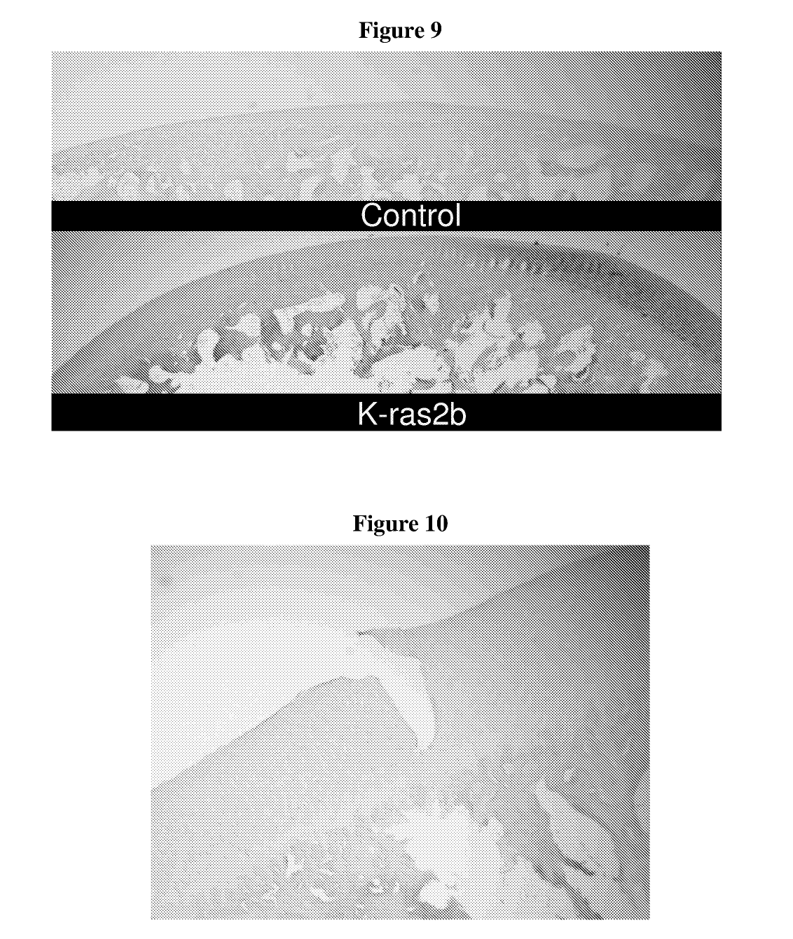

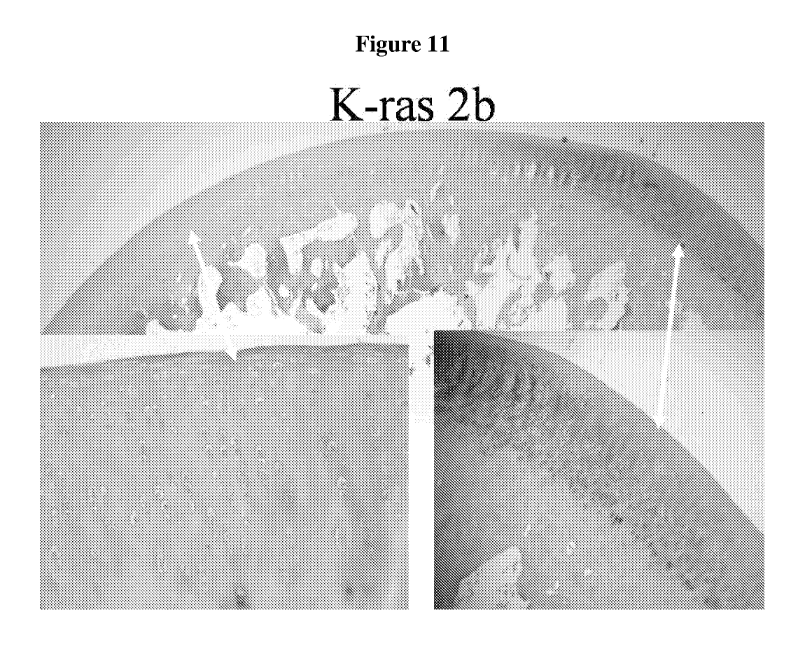

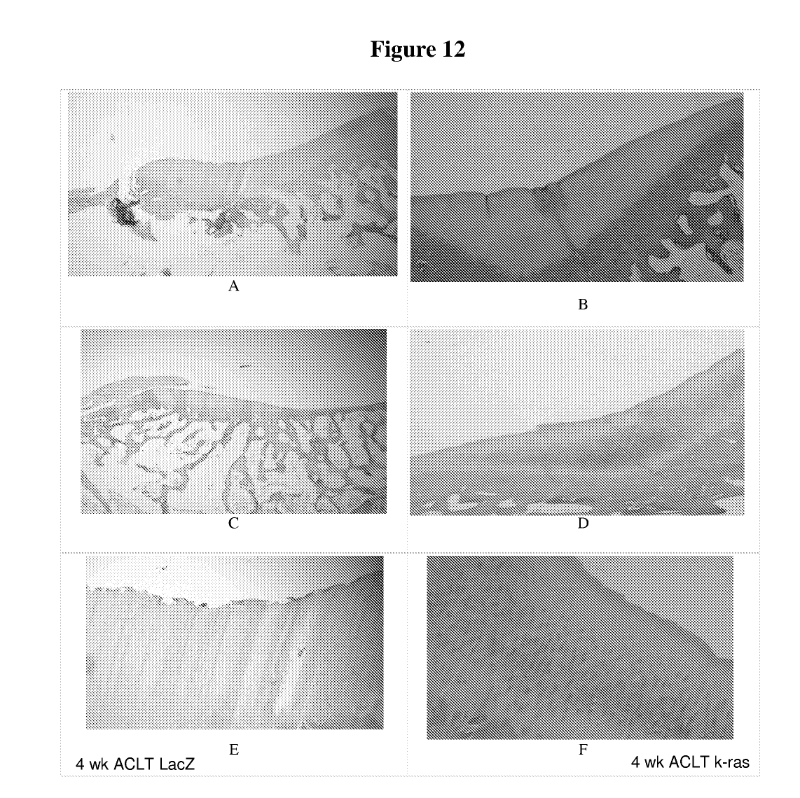

In addition to the macroscopic degradation discussed above, the cartilage of control (beta-galactosidase treated) knees showed that proteoglycan levels were reduced (as determined by reduced histological retention of safranin-O stain), cartilage height was significantly reduced and the cartilage cells (chondrocytes) were disorganized (FIG. 9 top panel, FIG. 10 and FIG. 11 (left panel)). Kras-2b expression prevented osteoarthritic degradation of the cartilage at 4 weeks ACLT. The Kras-2b transfected knees showed preserved proteoglycan levels as determined by safranin-O staining (FIGS. 9-12). In addition, the cartilage surface of the Kras-2b treated knees lacked significant lesions (FIG. 9-12) and the cells remained relatively well organized (FIG. 10).

These results demonstrate that changing the cellular proliferation index by delivering a wild-type tumor suppressor gene (Kras-2b) into the synovium of animals undergoing ACLT procedure prevented the articular cartilage from developing OA related pathological damage for the 4 week study period. Therefore, it appears that this gene product can protect cartilage in vivo. Additionally, the non-viral gene therapy protocol detailed here was successful in delivering genes in-vivo at a very high efficiency and was long-lasting (28 days). Kras-2b repressed cellular proliferation in articular synovium by attenuating S6 ribosomal transcripts in a dose dependent manner (FIG. 3). This is, presumably, one of the main mechanisms leading to its success in protecting cartilage from OA related degradation in our animal model. In addition, Kras-2b also increases the production of hyaluronic acid synthase (HAS) and is an inhibitor of COX-2 (Smakman & Kranenburg, (2005) Clinical Cancer Research, 11:41-48; Itano et al., (2004) J. Biol. Chem., 279:18679-87). It is assumed that other molecules in the signaling cascade of ras/raf/rac and Tob1 and/or mTOR may function similarly to protect cartilage.

Various particularly preferred embodiments of the present invention are described above and generally claimed below. The invention now being fully described herein, it will be apparent to those of ordinary skill in the art that many changes and modifications can be made to this invention without departing from the spirit or scope of the invention as presently claimed.

1. A composition for introducing an exogenous nucleic acid encoding Kras-2b into a target cell in the joint of a mammal, comprising
a synthetic liposome comprising an exogenous nucleic acid encoding Kras-2b, at least one ligand, and a poly-1-lysine polymeric scaffold,
wherein said polymeric scaffold is attached to both said ligand and said nucleic acid.

2. The composition of claim 1, wherein said liposome is selected from the group consisting of cationic liposomes, anionic liposomes, and synthetic lipid microspheres.

3. The composition of claim 1, wherein said ligand is transferrin.

4. The composition of claim 1, wherein said polymeric scaffold is selected from the group consisting of positively charged polymeric scaffolds, uncharged polymeric scaffolds, homopolymeric scaffolds, and poly-L-lysine scaffolds.

5. The composition of claim 1, wherein the target cell is a human cell.

6. The composition of claim 1, wherein said target cell is a perichondrial cell, cartilage cell, flexor tendon cell, tendon sheath cell, tendon pulley cell, or a skin cell.

7. The composition of claim 1, wherein said nucleic acid is contained within a vector.

8. A device containing a pharmaceutical composition comprising a therapeutic agent which is (i) one or more Kras-2b polypeptides or (ii) one or more polynucleotides capable of expressing one or more Kras-2b polypeptides in a target cell in the joint of a mammal, said device being suitable for intra-synovial delivery of said composition.

9. A method of protecting a joint from degradation in a mammal, comprising delivering a vector to target cells in the joint of said mammal, the vector comprising a gene encoding Kras-2b operably linked to a promoter.

10. The method of claim 9, wherein the vector is introduced intra-synovially, intramuscularly, subcutaneously and/or transdermally.

11. The method of claim 9, wherein the target cell is selected from the group consisting of primary perichondrial cells, cartilage cells, flexor tendon cells, tendon sheath cells, tendon pulley cells, epithelial skin cells, skin, muscle, fat, fibroblasts, dendritic cells, hair cells, keratinocytes, cells in the hair shaft, nerve cells, vascular smooth muscle cells, blood vessel walls, immune cells (B-cells, T-cells, Neutrophils, NTK cells, macrophages, mononuclear cells, osteoblasts, osteoclasts, liver cells, heart smooth muscles, bronchial aveolii, other pulmonary cells, buccal cavity cells, nasal cavity cells, mononuclear marrow cells, multinucleated marrow cells, spinal chord, motoneurons, ganglia, and cells of the CNS.

12. The method of claim 9, wherein said mammal is a human.

13. The method of claim 9, wherein the vector comprises a gene encoding Kras-2b as disclosed in Table I.

14. The method of claim 9, wherein the gene encoding Kras-2b is operably linked to a heterologous promoter selected from the group consisting of a heterologous constitutive promoter and a heterologous inducible promoter.

15. The method of claim 9, wherein prior to introduction of the vector, the target cells are permeabilized with a permeabilization agent selected from the group consisting of lysolecithin, polyoxyethylenesorbitan monolaurate, octylphenoxy polyethoxy ethanol, t-octylphenoxypolyethoxyethanol, phosphatidylcholine, phospholipase, osmotic shock, and electric pulses.

16. The method of claim 9, wherein the vector is attached to a polymeric scaffold which is further attached to a ligand, and wherein the attached ligand, scaffold, and vector are encapsulated in a synthetic liposome.

17. The method of claim 9, wherein the gene encoding Kras-2b is a variant of a wild-type Kras-2b gene.

18. The method of claim 9, wherein the vector is a non-viral vector.

19. The method of claim 9, wherein the method prevents and/or treats osteoarthritis.

20. A method of expressing Kras-2b in a cell of the joint of a mammal in vivo, comprising
introducing a vector encoding Kras-2b into a target cell in the joint of a mammal in vivo; and
maintaining the cell in vivo under conditions permitting expression of Kras-2b in the cell.

21. A method of treating a mammal with osteoarthritis, comprising introducing into the patient an expression vector encoding Kras-2b, such that an amount of Kras-2b effective to alleviate one or more symptoms of osteoarthritis is expressed in the mammal.

22. The method of claim 21, wherein the mammal is a human.

23. The method of claim 21, wherein the expression vector is introduced by intra-synovial injection.

24. The method of claim 21, wherein the expression vector is introduced two or more times.
